(19) United States
(12) Patent Application Publication
(10) Pub. No.: US 2024/0318107 A1
(43) Pub. Date: Sep. 26, 2024

(54) MICROBIOME MANAGEMENT IN AN ANIMAL RESIDENCE

(71) Applicant: SIGNIFY HOLDING B.V., EINDHOVEN (NL)

(72) Inventor: PETER DEIXLER, ARLINGTON, MA (US)

(21) Appl. No.: 18/578,037

(22) PCT Filed: Jul. 11, 2022
(86) PCT No.: PCT/EP2022/069324

§ 371 Date: Jan. 10, 2024

Related U.S. Application Data
(60) Provisional application No. 63/221,052, filed on Jul. 13, 2021.

(30) Foreign Application Priority Data
Jul. 26, 2021 (EP) 21187588.5

(51) Int. Cl. C12M 1/00; A01K 1/00; C12M 1/12; C12M 1/26; C12M 1/34; C12M 1/36

(52) U.S. Cl. CPC C12M 21/02; A01K 1/0047; C12M 33/00; C12M 37/00; C12M 41/06; C12M 41/30; C12M 41/48

(57) ABSTRACT

The invention provides a lighting system (1000) for indoor microbiome management in an animal residence (200), wherein the lighting system (1000) comprises a light generating device (100), a control system (300), and an input system (305), wherein: the light generating device (100) is configured to generate first device radiation (111), wherein a spectral power distribution of the first device radiation (111) is selected for promoting persistence of the first microbes (7) relative to second microbes, other than the first microbes (7); the input system (305) is configured to receive and/or sense a microbiome influencing parameter and to provide a related input signal to the control system (300): the control system (300) is configured to control the light generating device (100) in dependence of the related input signal.

### FIELD OF THE INVENTION

The invention relates to a lighting system for indoor microbiome management in an animal residence. The invention further relates to a lighting device comprising the lighting system. The invention further relates to an animal residence system comprising the lighting system.

### BACKGROUND OF THE INVENTION

Systems for monitoring and managing microbiomes in a facility are known in the art. For instance, US2017081707A1 describes an automated facility system comprising means for collecting and sequencing microbiome samples from the facility; means for measuring facility operation parameters; and means for automated modification of facility operation parameters in response to detection of nucleotide sequences that fall within a predetermined sequence identity definition; wherein the facility operation parameters are modified to optimize facility performance on an ongoing basis as sequence data is obtained from the samples. Moreover, US2016030609A1 discloses a disinfecting lighting fixture.

### SUMMARY OF THE INVENTION

All animals may be home to bacteria, archaea, fungi, microbial eukaryotes, the collection of which may often be referred to as the microbiome. Faster and cheaper DNA sequencing and data analysis pipelines over the past two decades may have enabled a surge of investigations into the microbiome and its role in animal health and disease, including host nutrition, metabolism, development, immune function, and behavior. Historically, research may have primarily focused on problematic/undesirable microbes, such as Salmonella and Streptococcus species. However, the animal residence environment may comprise rich ecologies of fast-evolving microbe populations. These microbe populations may, however, have little overlap with outdoor microbe populations, including salutary species that animals co-evolved with over millions of years. Hence, the prior art may describe the disinfection of animal residence environments (and objects) to reduce exposure to pathogens, and the use of antibiotics to combat potential infections.

The common use of antibiotics in animal residences may have been found to have far reaching effects on the animal's beneficial microbes beyond their impact on the pathogen(s) they are administered to treat. With increasing pressure to reduce antibiotic use in foods, incentives to leverage the microbiome as a tool to promote healthier and more productive livestock may now be greater than ever. The microbiome in an animal residence may be directly or indirectly affected by a variety of factors, including the animals residing in the animal residence, the animal density in the animal residence, the frequency of humans being present, including food present in the animal residence, including bedding materials, and including environmental parameters, and the microbiome itself may subsequently impact the animal (wellbeing).

For instance, with regards to (growing) pigs, the rearing-environment may impact the barn-animal's microbial community. Modern animal residence may typically lack natural bacterial reservoirs, which strongly affects the microbial structure, diversity and function of microbiota. In particular, the Environment-to-Animal microbiome exchange may be important for the early microbial colonization of pigs. Further, also the environmental complexity in the animal residence may impact the respiratory and gut microbiome community structure and diversity. While the first inoculum for microbiota assembly for just born pigs may be maternal-dependent, the subsequent progression may be substantially environmental-dependent. The environmentally-acquired microbes may be essential building blocks of an animal's microbial community; hence any deleterious shift in the physical environment (e.g. anti-biotics or excessive disinfection or non-optimal rearing environment) can substantially disrupt the composition and functionality of the animal microbial community during the animal's growing period. In particular, limiting microbial exposure during the animal development, such as by maintaining animals in environments of excessive hygiene, may have a negative impact on the composition and dynamics of the adult pig microbiota. Indoor-raised piglets may generally have substantially reduced microbial diversity and richness, such as ileal mucosa-adherent microbial diversity and richness, compared to outdoor-raised piglets.

The prior art may further describe solutions based on competitive exclusion to reduce the prevalence of pathogens in animals and/or their environment. Such prior art solutions may, however, be limited in scope (targeting a specific pathogen), may be cumbersome, such as time-intensive, in use, and may be unsuitable for dynamically adapting to changing (environmental) conditions.

Hence, it is an aspect of the invention to provide an alternative system for microbiome management in an animal residence, which preferably further at least partly obviates one or more of above-described drawbacks. The present invention may have as object to overcome or ameliorate at least one of the disadvantages of the prior art, or to provide a useful alternative.

Hence, in a first aspect, the invention may provide a system, especially a lighting system, for indoor microbiome management in an animal residence. The system may comprise a light generating device, a control system, a microbiome dispenser device, and an input system, especially a sensor system. The microbe dispenser device may be configured to provide in a microbic emission mode an emission of the first microbes. The system, especially the control system, may have a microbic lighting mode. In the microbic lighting mode, the light generating device may be configured to generate first device radiation, wherein a spectral power distribution of the first device radiation is selected for promoting persistence, especially growth, of first microbes relative to second microbes, other than the first microbes. In embodiments, the input system may be configured to receive and/or sense a microbiome influencing parameter and to provide a related input signal to the control system. In further embodiments, the control system may be configured to control the microbic lighting mode, especially the light generating device, in dependence of the related input signal.

In particular, the invention may provide a system for an animal residence configured to provide suitable lighting conditions for growth of beneficial microbes in the animal residence, such as in a rearing space of the animal residence. The lighting system may, for instance, specifically target building surfaces and objects in the animal residence and make them a bacterial reservoir for desired but usually relatively rare or transient “good” microbes, such as good bacteria. Hence, the administering of suitable light conditions tends a healthy microbial community within an animal's immediate (rearing) environment; the presence of a large and diverse bacteria reservoir with rare/transient microbe species may lead to an increased competition with the core microbes in the animal residence, and may facilitate suppressing undesired second microbes.

Hence, the (lighting) system of the invention may provide the benefit that a microbial composition of a microbiome in an animal residence may be steered using the first device radiation, especially in view of entered (via a user interface), measured, inferred, and/or retrieved microbiome influencing parameters. Thereby, animals may be exposed to a desirable microbiome, which may positively impact various aspects of animal wellbeing, including with regards to digestive and respiratory characteristics, as well as with regards to overall health and temperature management, which may in turn positively affect animal performance, such as with regards to weight gain, or such as with regards to productivity.

In particular, the zest of the invention may be to selectively promote a first microbe (which, for instance may be known to be relatively robust against a selected light wavelength) vs a second microbe which may be more susceptible to the selected wavelength, such as 405 nm. The first microbe may also be damaged or deactivated by the selected wavelength, but to a lesser degree than the second microbe, such as due to a lower absorption of radiation at the selected wavelength, or such as due to better or more active repair mechanisms. Hence, the selected wavelength may shift the balance between the first and second microbe, helping the first microbe to dominate (relative to the second microbe) in the animal residence via a competitive exclusion mechanism.

In specific embodiments, the invention may provide a lighting system for indoor microbiome management in an animal residence, wherein the lighting system comprises a light generating device, a control system, and an input system, wherein: the light generating device is configured to generate first device radiation, wherein a spectral power distribution of the first device radiation is selected for promoting persistence of the first microbes relative to second microbes, other than the first microbes; the input system is configured to receive and/or sense a microbiome influencing parameter and to provide a related input signal to the control system; and the control system is configured to control the light generating device in dependence of the related input signal.

In embodiments, the invention may provide a system, especially a lighting system, for indoor microbiome management in an animal residence.

The term “indoor microbiome” may herein refer to the collection of microbes in a built environment, especially in an indoor environment. In general, the indoor microbiome may comprise a plurality of different species, including bacteria, archaea, and fungi. In particular, an indoor environment may host a plurality of different indoor microbiomes together forming a total indoor microbiome. For instance, the microbiome on a feeding trough surface may differ from that of a floor surface, and both may differ from that on a wall surface.

In embodiments, the indoor microbiome may especially be a surface indoor microbiome, such as the microbiome of one or more of a feeding trough, a floor, a wall, or a ceiling.

In further embodiments, the indoor microbiome may be a (total) room indoor microbiome, i.e., the indoor microbiome may comprise all microbes in an (open) room.

The term “microbiome management” may in embodiments amongst others include controlling one or more of the spectral power distribution of the first device radiation and the radiant flux of the first device radiation. In embodiments, the radiation flux may e.g. be controlled by controlling a duty cycle of the first device radiation, though other options may also be possible. Further, other ways of microbiome management are herein not excluded (see also below).

The term “animal residence” may herein especially refer to any accommodation suitable for the keeping of animals, especially livestock. Generally, an animal may be any structure, especially (in) a building, configured to house livestock. The animal residence may comprise an indoor space. The indoor space may especially be configured for housing the animals, especially the livestock. In embodiments, the indoor space may comprise stalls. Additionally or alternatively, the indoor space may comprise (specialized) farm equipment such as feeding equipment and/or milking equipment. In embodiments, the indoor space may comprise a rearing space. In further embodiments, the animal residence may comprise one or more of a stable, a barn, a shed, and a pen.

Hence, in embodiments, the animal residence may be configured for the keeping of livestock, especially of pigs, or especially of cattle.

The term “livestock” (also: “animal”) may herein generally refer to any wild or domesticated animal raised in an agricultural setting to produce an animal product. The term “livestock” especially refers to any farm animal, such as cattle, including cows, sheep, goats, pigs, horses, fish and/or poultry. The term livestock may further refer to any animal that is kept to provide an animal product, including animals such as worms and insects providing alternative protein sources. Livestock especially refers to ruminants, more especially to cattle, such as cows.

In embodiments, the system may further comprise a light generating device. The term “light generating device” may especially refer to a device configured to provide (visible) light. In embodiments, the light generating device may be selected from the group of a lamp, a luminaire, a projector device, and a (UV and/or IR) disinfection device.

Hence, the system may especially be a lighting system.

The light generating device may especially be configured to (in a microbic lighting mode of the system) provide first device radiation, especially a first beam of first device radiation. In particular, the first device radiation may have a spectral power distribution selected for promoting persistence of first microbes, especially relative to second microbes, other than the first microbes.

The term “persistence” may herein especially refer to the continued presence of the first microbes, especially relative to the second microbes. Hence, the first device radiation may have a spectral power distribution selected for promoting the presence of the first microbes, especially relative to the second microbes. In particular, in embodiments, the first device radiation may have a spectral power distribution selected for positively affecting the first microbes, such as promoting growth of the first microbes, especially relative to the second microbes. Thereby, the first microbes may accumulate in the animal residence, such as on a surface, or such as in an indoor space. In further embodiments, the first device radiation may have a spectral power distribution selected for negatively affecting the second microbes, such as deactivating the second microbes, especially diminishing growth of the second microbes. Thereby, the second microbes may diminish in the microbiome in the animal residence, such as on a surface, or such as in an indoor space, which may diminish, especially remove, a competitor for the first microbes.

In particular, the first device radiation may have a spectral power distribution selected to provide a competitive advantage to the first microbes relative to the second microbes, i.e., to promote the persistence of the first microbes relative to the second microbes. A competitive advantage may be provided by providing a larger benefit (or smaller detriment) to the first microbes relative to the second microbes. Thereby, the first microbes may persist in the animal residence, especially (over time) become more prevalent in the animal residence, with respect to the second microbes.

Hence, in embodiments, the first device radiation may have a spectral power distribution selected to deactivate second microbes more strongly than the first microbes.

In further embodiments, the spectral power distribution of the first device radiation may be selected for one or more of (i) promoting growth of the first microbes, (ii) deactivating (or “diminishing growth of the”) second microbes, other than the first microbes, (iii) deactivating second microbes more strongly than the first microbes, and (iv) deactivating viruses.

For example, light may promote the growth of microbes, especially also of heterotrophic microbes, such as described in Fahimipour et al., “Daylight exposure modulates bacterial communities associated with household dust”, Microbiome, 2018, which is hereby herein incorporated by reference, which may specifically describe that while daylight may usually be associated with the loss of microbes, it may also lead to increases in the abundances of some specific microbes. The paper may further demonstrate that light exposure per se led to lower abundances of viable bacteria and communities that were compositionally distinct from dark rooms, suggesting preferential inactivation of some microbes over others under daylighting conditions. Hence, light may be used to (i) promote growth, (ii) cause loss of microbes, and (iii) to cause more substantial loss for second microbes with respect to first microbes. Further, Maresca et al., “Light Modulates the Physiology of Nonphototrophic Actinobacteria”, Journal of Bacteriology, 2019, which is hereby herein incorporated by reference, may describe that Actinobacteria, specifically strains Rhodoluna lacicola MWH-Ta8 and Aurantimicrobium sp. MWH-Uga, grew faster in blue light and UV light, but not in red or green light. Hence, the light exposure may have promoted the growth of these Actinobacteria.

The first microbes (or: “first micro-organisms”) may especially comprise beneficial microbes, including microbes with (direct) health benefits, but also microbes that may compete with pathogenic (or otherwise undesirable) microbes and may thereby (indirectly) provide health benefits.

In embodiments, the first microbes may be selected from the phylum Firmicutes, especially from the class Bacilli, such as from the order of Lactobacillales, especially from the family of Lactobacillaceae, such as from the genus Lactobacillus. In such embodiments, the second microbes may especially be selected from the genera Staphylococcus and Vibrio.

The first microbes may be selected for providing a (direct or indirect) positive effect, include via competitive exclusion, on animals, especially livestock animals, such as for pigs.

Hence, in embodiments, the first microbes may be selected from the group comprising the genera Acinetobacter, Alcaligenes, Arthrobacter, Azospirillum, Azotobacter, Bacillus, Beijerinckia, Caldiarchaeum, Cenarchaeum, Deinococcus, Enterobacter, Erwinia, Flavobacterium, Lactobacillus, Nitrosoarchaeum, Nitrosocaldus, Nitrosomonas, Nitrosopumilus, Nitrosospira, Rhizobium and Serratia. The first microbes may especially comprise a plurality of different species, such as different genera. The terms “first microbe”, or “first microbes”, and similar terms, may refer to one or more different types of microbes. In further embodiments, the first microbes may comprise (species belonging to) two or more of the genera Acinetobacter, Alcaligenes, Arthrobacter, Azospirillum, Azotobacter, Bacillus, Beijerinckia, Caldiarchaeum, Cenarchaeum, Deinococcus, Enterobacter, Erwinia, Flavobacterium, Lactobacillus, Nitrosoarchaeum, Nitrosocaldus, Nitrosomonas, Nitrosopumilus, Nitrosospira, Rhizobium and Serratia. Hence, the first microbes may comprise a microbial community of two or more species. In further embodiments, the first microbes may be selected from the group comprising the species of Bacillus subtilis, Bacillus amyloliquefaciens, Lactobacillus gasseri, Lactobacillus reuteri, Lactobacillus fermentum, and Lactobacillus acidophilus. In specific embodiments, the first microbes may comprise (at least) a Lactobacillus spp., such as one or more of Lactobacillus gasseri, Lactobacillus reuteri, Lactobacillus fermentum, and Lactobacillus acidophilus.

The first microbes may further be selected for providing a (direct or indirect) positive effect on humans, include via competitive exclusion, such as for people working in the animal residence.

In further embodiments, the first microbes may be selected from the group comprising the genera Akkermansia, Bifidobacterium, Lachnospira, Alistipes, Bacteroides, Coprococcus, Viellonella, Faecalibacterium, Roseburia, Dialister, Sutterella, Methanobrevibacter, Lactobacillus, Parabacteroides, Prevotella, Agathobacter, Eubacterium, Ruminococcus, Nitrosomonas, Nitrosospira, Nitrosopumilus, Cenarchaeum, Nitrosoarchaeum, Nitrosocaldus, Caldiarchaeum, Acinetobacter, Alcaligenes, Arthrobacter, Azospirillum, Azotobacter, Bacillus, Beijerinckia, Enterobacter, Erwinia, Flavobacterium, Rhizobium, Serratia and Deinococcus. In further embodiments, the first microbes may comprise (species belonging to) two or more of the genera Akkermansia, Bifidobacterium, Lachnospira, Alistipes, Bacteroides, Coprococcus, Viellonella, Faecalibacterium, Roseburia, Dialister, Sutterella, Methanobrevibacter, Lactobacillus, Parabacteroides, Prevotella, Agathobacter, Eubacterium, Ruminococcus, Nitrosomonas, Nitrosospira, Nitrosopumilus, Cenarchaeum, Nitrosoarchaeum, Nitrosocaldus, Caldiarchaeum, Acinetobacter, Alcaligenes, Arthrobacter, Azospirillum, Azotobacter, Bacillus, Beijerinckia, Enterobacter, Erwinia, Flavobacterium, Rhizobium, Serratia and Deinococcus. In further embodiments, the first microbes may be selected from the group comprising the species RF39, Akkermansia mucinophila, Bifidobacterium longum, Roseburia inulinivorans and Faecalibacterium prausnitzii.

Hence, in further embodiments, the first microbes may be selected from the group comprising the species Bacillus subtilis, Bacillus amyloliquefaciens, Lactobacillus gasseri, Lactobacillus reuteri, Lactobacillus fermentum, Lactobacillus acidophilus, RF39, Akkermansia mucinophila, Bifidobacterium longum, Roseburia inulinivorans and Faecalibacterium prausnitzii.

The term “second microbes” may herein refer to microbes other than the first microbes. Specifically, the first microbes may be desirable microbes in the indoor space, whereas the second microbes may be undesirable due to their effect on humans, animals and/or on the first microbes. In embodiments, the second microbes may be selected from the group comprising the genera Actinobacillus, Actinobacteria, Aeromonas, Bacillus, Bordetella, Brachyspira, Campylobacter, Clostridium, Corynebacterium, Erysipelothrix, Haemophilus, Lawsonia, Legionella, Leptospira, Listeria, Mycoplasma, Neisseria, Pasteurella, Plesiomonas, Psychrobacter, Salmonella, Shigella, Staphylococcus, Streptococcus, Vibrio, and Yersinia. In further embodiments, the second microbes may be selected from the group comprising the species Actinobacillus pleuropneumoniae, Actinobacillus suis, Actinomyces spp., Aeromonas hydrophila, Bacillus anthracis, Bacillus cereus, Bordetella bronchiseptica, Bordetella pertussis, Borrelia burgdorferi, Brachyspira hyodysenteriae, Brachyspira murdochii, Brachyspira pilosicoli, Brucella abortus, Brucella melitens, Brucella suis, Campylobacter fetus, Campylobacter jejuni, Chlamydia pneumoniae, Chlamydia psittaci, Chlamydia trachomatis, Clostridium botulinum, Clostridium difficile, Clostridium perfringens, Clostridium tetani, Corynebacterium diptheriae, Enterococcus faecalis, Erysipelothrix rhysiopathiae, Escherichia coli, Francisella tularensis, Haemophilus influenzae, Haemophilus parasuis, Helicobacter pylori, Lawsonia intracellularis, Legionella pneumophila, Leptospira interrogans, Listeria monocytogenes, Mycobacterium leprae, Mycobacterium tuberculosis, Mycoplasma hyopneumoniae, Mycoplasma hyorhinis, Mycoplasma hyosynoviae, Mycoplasma pneumoniae, Neisseria gonorrhoeae, Neisseria meningitidis, Nocardia spp., Pasteurella multocida, Plesiomonas shigelloides, Pseudomonas aeruginosa, Pseudomonas mallei, Rickettsia akari, Rickettsia prowazekii, Rickettsia ricketsii, Salmonella enterica, Salmonella enteritidis, Salmonella Typhimurium, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus suis, Treponema pallidum, Ureaplasma urealyticum, Vibrio cholerae (O1), Vibrio parahaemolyticus, Vibrio vulnificus, Yersinia enterocolitica, Yersinia pestis, Yersinia pseudotuberculosis.

For instance, in embodiments, the first microbes may comprise a Lactobacillus plantarum, and the second microbes may comprise Vibrio parahaemolyticus. When illuminated with first device radiation comprising wavelengths in the range of 400-410 nm, especially about 405 nm, or with first device radiation comprising wavelengths in the range of 455-465 nm, especially about 405 nm, V. parahaemolyticus may be substantially inactivated, while L. plantarum was substantially less susceptible to the illumination. Hence, illumination with wavelengths of about 405 or about 460 nm may promote the persistence of L. plantarum with respect to V. parahaemolyticus.

In terms of genera, there may be overlap between the first microbes and the second microbes. For instance, a genus may comprise both a desirable species and an undesirable species, such as Bacillus subtilis and Bacillus anthracis. Further, the desirability of a microbe may, for instance, also depend on the animal, such as depend on an age of the animal, or such as depend on a health status of the animal. For example, a microbe may be generally harmless, but may be detrimental to an animal with a compromised immune system, or may be detrimental if present in relatively large (relative) abundance.

In further embodiments, the first microbes may be selected from the genera Akkermansia, Bifidobacterium, Lachnospira, Alistipes, Bacteroides, Coprococcus, Viellonella, Faecalibacterium, Roseburia, Dialister, Sutterella, Methanobrevibacter, Lactobacillus, Parabacteroides, Prevotella, Agathobacter, Eubacterium, Ruminococcus, Nitrosomonas, Nitrosospira, Nitrosopumilus, Cenarchaeum, Nitrosoarchaeum, Nitrosocaldus, Caldiarchaeum, Acinetobacter, Alcaligenes, Arthrobacter, Azospirillum, Azotobacter, Bacillus, Beijerinckia, Enterobacter, Erwinia, Flavobacterium, Rhizobium, Serratia and Deinococcus. The second microbes may be selected from the genera Actinobacteria, Aeromonas, Campylobacter, Clostridium, Corynebacterium, Listeria, Neisseria, Plesiomonas, Psychrobacter, Salmonella, Shigella, Staphylococcus, Streptococcus, Vibrio, and Yersinia.

Hence, the term “first microbes”, and similar terms, may especially refer to (desirable) bacteria. Instead of the term “first microbes”, also the term probiotics may be applied. The term “probiotics” herein may in embodiments especially refer to live bacteria or yeasts that are good for an animal. Note that the term “probiotics” does not necessarily refer to microbes that enter the gastro-intestinal tract. The term probiotics may also refer to microbes that may be good for the skin or fur, or may be bad for undesirable microbes (second microbes). The term “second microbes”, and similar terms, may especially refer to bacteria that are not desired. The term “second microbes”, and similar terms, may also refer to viruses.

The term “first microbes” may in embodiments refer to a plurality of different first microbes. The term “second microbes” may in embodiments refer to a plurality of different second microbes.

In further embodiments, the first device radiation (or the disinfection radiation; see below) may be configured to remove (or “deactivate” or “kill”) a virus (as second microbe). In embodiments, the virus (as second microbe) may comprise an animal pathogen. In further embodiments, the virus (as second microbe) may be a microbial pathogen, such as a virus infecting the first microbes. For instance, in embodiments, the virus (as second microbe) may comprise a bacteriophage, such as a bacteriophage targeting one or more of the first microbes.

In embodiments, the viruses, especially the second viruses, may comprise an animal pathogen, especially an animal pathogen for the animal kept in the animal residence, i.e., the animal residence may be configured to host an animal, and the virus may be a pathogen for the animal. For instance, especially in embodiments wherein the animal residence is configured for hosting pigs, the viruses, especially the second viruses, may comprise one or more of porcine adenovirus 1, porcine adenovirus 2, porcine adenovirus 3, African swine fever virus, Porcine astrovirus 1, Porcine respiratory and reproductive syndrome virus, Vesicular exanthema of swine virus, Porcine sapovirus, Porcine circovirus 2, Porcine circovirus 3, Transmissible gastroenteritis virus, Porcine respiratory coronavirus, Porcine hemagglutinating encephalomyelitis virus, Porcine deltacoronavirus, Porcine Epidemic Diarrhea Virus, Reston virus, Japanese encephalitis virus, Classical swine fever virus, Atypical porcine pestivirus, Pseudorabies virus, Porcine cytomegalovirus, Hepatitis E virus, Influenza A virus in swine, Influenza B, Influenza C, Influenza D, Swine papillomavirus, Menangle virus, Blue eye paramyxovirus, Nipah virus, Porcine parainfluenza virus 1, Sendai virus, Porcine parvovirus, Porcine parvovirus 1, Porcine parvovirus 2, Porcine parvovirus 3, Porcine parvovirus 4, Porcine parvovirus 5, Porcine parvovirus 6, Porcine parvovirus 7, Foot-and-mouth disease virus, Encephalomyocarditis virus, Coxsackievirus B4, Coxsackievirus B5, Porcine kobuvirus, Porcine sapelovirus, Seneca Valley virus, Porcine teschovirus, Swinepox virus, Rotavirus A, Rotavirus B, Rotavirus C, Rotavirus E, Rotavirus H, Porcine reovirus, Getah virus, Chikungunya virus, Vesicular stomatitis virus, Vesicular stomatitis Indiana virus, Vesicular stomatitis New Jersey virus, and Rabies virus.

Here above, the viruses are in general considered as second microbe, i.e. a less or no desired microbe. However, in specific embodiments there may also be viruses, like bacteriophages, that may be detrimental to some bacteria as second microbes. Such viruses, detrimental to second microbes may also be indicated as (embodiment of) first microbes. Hence, in embodiments the first microbes may also comprise bacteriophages.

Some bacteriophages are viruses are harmless to people but they attack harmful microbes. Such bacteriophages may promote persistence of first microbes relative to second microbes. Hence, the first microbe (directly or indirectly) create a positive health impact on humans. Therefore, some bacteriophages (as they may attacks second microbes) may have a (indirectly) positive impact on humans as it deactivates a harmful second microbe. Hence, in embodiments a lighting setting may be chosen such that the bacteriophage as first microbe may be selectively promoted versus the second microbe. Consequently, as the light may deactivate the bacteriophage less, the bacteriophage as first microbe can be more detrimental to the second microbe.

Especially, microbes classified as first microbes are no second microbes and microbes classified as second microbes are no first microbes.

The spectral power distribution of the first device radiation may be selected in view of the wavelength-dependent sensitivities of the first microbes and the second microbes (and optionally the virus). For instance, the wavelength-dependent Log 10 reduction dose for the microbes (and the virus) may be considered. Some examples of Log 10 doses for swine pathogens are, for instance, described in the white paper Wedel, Johnson, “Ultraviolet C (UVC) Standards and Best Practices for the Swine Industry”, swinehealth.org, 19 Oct. 2020, and in Malayeri et al., “Fluence (UV Dose) Required to Achieve Incremental Log Inactivation of Bacteria, Protozoa, Viruses and Algae”, UV Solutions, 2016, which are hereby herein incorporated by reference.

For instance, in embodiments, the first microbes may be selected from the genera Flavobacterium and Rhizobium, whereas the second microbes may comprise Staphylococcus aureus, Shigella dysenteriae and/or Escherichia coli, and the spectral power distribution may comprise UVC as Flavobacterium spp. and Rhizobium spp. may have a substantially higher log 10 (kill) dose in UVC as compares to S. aureus, S. dysenteriae and E. coli.

For example, light may promote the growth of microbes, especially also of heterotrophic (first) microbes, such as described in Fahimipour et al., “Daylight exposure modulates bacterial communities associated with household dust”, Microbiome, 2018, which is hereby herein incorporated by reference, which may specifically describe that while daylight may usually be associated with the loss of microbes, it may also lead to increases in the abundances of some specific microbes. The paper may further demonstrate that light exposure per se led to lower abundances of viable bacteria and communities that were compositionally distinct from dark rooms, suggesting preferential inactivation of some microbes over others under daylighting conditions. Hence, light may be used to (i) promote growth, (ii) cause loss of microbes, and (iii) to cause more substantial loss for second microbes with respect to first microbes. Further, Maresca et al., “Light Modulates the Physiology of Nonphototrophic Actinobacteria”, Journal of Bacteriology, 2019, which is hereby herein incorporated by reference, may describe that Actinobacteria, specifically strains Rhodoluna lacicola MWH-Ta8 and Aurantimicrobium sp. MWH-Uga, grew faster in blue light and UV light, but not in red or green light. Hence, the light exposure may have promoted the growth of these Actinobacteria.

In embodiments, the light generating device may be configured to provide the first device radiation to one or more of a floor of the animal residence, a wall of the animal residence, a feeding element, such as a trough, in the animal residence, and a sleeping part, such as a straw bed, in the animal residence.

In embodiments, the system, especially the control system (see below), may have a microbic lighting mode. Especially, in the microbic lighting mode, the light generating device may be configured to provide the first beam of the first device radiation. In particular, in the microbic lighting mode, the spectral power distribution of the first device radiation may be selected for providing a competitive advantage to the first microbe(s) relative to second microbe(s), other than the first microbe(s).

In embodiment, the system may have a control system. The control system may especially be configured to control one or more elements of the system. The term “controlling” and similar terms herein may especially refer at least to determining the behavior or supervising the running of an element. Hence, herein “controlling” and similar terms may e.g. refer to imposing behavior to the element (determining the behavior or supervising the running (or “activity”) of an element), etc., such as e.g. measuring, displaying, actuating, opening, shifting, changing temperature, etc. . . . Beyond that, the term “controlling” and similar terms may additionally include monitoring. Hence, the term “controlling” and similar terms may include imposing behavior on an element and also imposing behavior on an element and monitoring the element. The controlling of the element can be done with a control system. The control system and the element may thus at least temporarily, or permanently, functionally be coupled. The element may comprise the control system. In embodiments, the control system and the element may not be physically coupled. Control can be done via wired and/or wireless control. The term “control system” may also refer to a plurality of different control systems, which especially are functionally coupled, and of which e.g. one master control system may be a control system and one or more others may be slave control systems.

In further embodiments, the system may comprise an input system, especially a sensor. The input system may especially comprise one or more of a sensor, a user interface, and a data retrieval system. In embodiments, the input system may be configured to receive and/or sense a microbiome influencing parameter, and especially to provide a related input signal to the control system.

Hence, in embodiments, the input system may comprise a sensor system, wherein the sensor is configured to sense the microbiome influencing parameter, and wherein the related input signal comprises a related sensor signal. In further embodiments, the sensor may be selected from the group comprising a movement sensor, a presence sensor, an activity detection sensor, an animal counting sensor, a distance sensor, an ion sensor, a gas sensor, a volatile organic compound (VOC) sensor, a pathogen sensor, an airflow sensor, a sound sensor, a temperature sensor, and a humidity sensor. In further embodiments, the sensor may be configured to detect an animal in the animal residence. In further embodiments, the control system may be configured to determine the location of an animal in the animal residence, and/or especially an activity of an animal in the animal residence, based on the related sensor signal. In further embodiments, the sensor may be configured to detect the presence of a human in the animal residence. In further embodiments, the control system may be configured to determine the location of a human in the animal residence, and/or especially an activity of a human in the animal residence, based on the related input signal, especially based on the related sensor signal. For instance, the sensor may detect a human performing a cleaning activity in the stable such as removing manure or power washing in the stable. The system may generate a heat map of areas in the stable occupied frequently by humans and for instance assign the lighting conditions such that a second microbe harmful to humans is suppressed in those areas.

In further embodiments, the sensor may be a radiofrequency receiver for receiving an entry comprising, or indicative of, the microbiome influencing parameter.

In further embodiments, the input system may comprise a user interface, wherein the user interface is configured to receive user input on a microbiome influencing parameter from a user, and wherein the related input signal comprises a related user input signal.

In further embodiments, the input system may comprise a data retrieval system, wherein the data retrieval system is configured to retrieve data on a microbiome influencing parameter from an (external) database, and wherein the related input signal comprises a related database signal.

The term related input signal may herein refer to a signal that is related to the received/detected input, such as to the microbiome influencing parameter. In particular, the related signal may comprise raw and/or processed data related to the (received/detected) input. Hence, the related sensor signal may especially comprise raw and/or processed data related to a sensed microbiome influencing parameter.

The term “microbiome influencing parameter” may herein especially refer to a parameter that may influence the (development of the) microbiome in the animal residence. In embodiments, the microbiome influencing parameter may comprise one or more of an animal-related parameter, a flooring-related parameter, a food-related parameter, a microbial presence-related parameter, and an environmental parameter. For instance, the microbiome influencing parameter may relate to one or more of (a) a floor type in the animal residence (e.g., concrete slatted-floor vs plastic flooring vs steel & cast swine flooring vs complex straw-based rearing ecosystem), (b) a type of bedding (wood shavings, straw, shredded paper), (c) a change of bedding type (e.g. straw and hay only used for sows but not for growers or finishers), (d) excrement on the bedding (e.g. poo/pee), (e) (excessive) disinfection within the animal residence, such as UV and ionizers in the animal residence (right now and over the history over recent weeks), (f) a presence of animals (for instance, the first device radiation may be applied only after surfaces have been disinfected, dried, and before animals re-enter the housing structure), (g) a type of feed (animal feed may influence the type of microbes on building surfaces e.g. haylage feed vs dry hay vs soaked hay), (h) recently administered antibiotics treatments in the animal residence (i.e. do they need a restoration of microbiome), (i) a spatial density of animals in the animal residence, (j) a health status of the animal population in the animal residence (e.g., to use microbiome to strengthen the animal's resistance to future infections), (k) a growth stage of an animal (e.g., weaning phase vs non-weaning phase), (l) a frequency, severity and type of infections currently/recently present in the animal residence, (m) a temperature and humidity of the animal residence (which may influence first microbes differently from second microbes), (n) whether the animals currently or recently have/had outdoor access, (o) a surface material to be colonized by the first microbe (the microbe growth may depend on the surface material, wetness) (p) a state of the animal, such as a sleeping stage, (e.g., if a strong 460 nm light pulse is administered to create a growth delta between the Lactobacilli, as example first microbes, and Vibrio parahaemolyticus, as example second microbes, this may preferably be done when the eyes of the animals are closed to limit impact on the circadian rhythm of the animal.

Similarly, first device radiation of about 405 nm may (strongly) deactivate the second microbe Listeria monocytogenes, while exposure to wavelengths longer than 450 nm may not substantially deactivate this microbe. Analysis of 10 nm bandwidths between 400 nm and 450 nm confirmed 405(±5) nm light to be most effective for the inactivation of L. monocytogenes, with a lesser bactericidal effect also evident at other wavelengths between 400 nm and 440 nm. Hence, first device radiation comprising a wavelength of 405 nm may promote the persistence, especially growth, of Lactobacillus plantarum compared to both Listeria monocytogenes and V. Parahaemolyticus, while a wavelength choice of 460 nm may substantially not affect the growth of both Lactobacillus plantarum and Listeria monocytogenes, while reducing the growth of Vibrio parahaemolyticus. Hence, the system, especially the control system, may control the light generating device to specifically influence the microbiome (composition) independence of the related input signal, especially such as determined by a sensor, to dynamically select which first microbes are to be promoted by the administered first device radiation at the expense of which second microbes. As the effectiveness of the light's interaction with microbes may (gradually) change with the administered wavelength, the control system may (be configured to) select the spectral power distribution of the first device radiation in view of a relative tradeoff between the currently present wanted microbes and unwanted microbes within the animal residence.

Hence, in embodiments, the spectral power distribution (of the first device radiation) may have an intensity at one or more wavelengths selected from a first wavelength range of 405 nm+/−5 nm or from a second wavelength range of 460+/−5 nm.

The light generating device may especially also serve as (normal) room lighting, such as animal housing lighting. Hence, the light generating device may have a dual function, both providing lighting for normal use of the animal housing, and providing the first device radiation for cultivating a desired microbiome in the room.

Hence, in embodiments, the system, especially the control system, may have a standard lighting mode. In further embodiments, in the standard lighting mode the light generating device may be configured to provide (standard) white (first) device radiation, especially to provide (standard) white light. In particular, the standard lighting mode may comprise providing general lighting, spot lighting, and wall washing. The term “standard lighting” may herein especially refer to lighting devoid of enriched light used to selectively promote desirable microbes.

The first device radiation provided during the microbic lighting mode may, in embodiments, also be suitable for normal indoor use. Specifically, in further embodiments, the light generating device is configured to provide in the microbic lighting mode (microbic) white first device radiation, especially (microbic) white light.

Hence, in further embodiments, the light generating device may be configured to provide (i) white first device radiation in a standard lighting mode, and (ii) white first device radiation in a microbic lighting mode. In such embodiments, a relative spectral power distribution of a first wavelength range relative to the spectral power distribution in the wavelength range of 200-780 nm may be at least 30% higher during at least part of the microbic lighting mode than during at least part of the standard lighting mode. In further embodiments, the first wavelength range may comprise the range of 405 nm+/−5 nm. In further embodiments, the first wavelength range may comprise the range of 460 nm+/−10 nm.

In further embodiments, a relative spectral power distribution of a first wavelength range of 400-400 nm, especially 400-420 nm, relative to the spectral power distribution in the wavelength range of 180-780 nm, especially 200-750 nm, may be higher during at least part of the microbic lighting mode than during at least part of the standard lighting mode, especially at least 30% higher, such as at least 60% higher, especially at least 100% higher.

In further embodiments, a relative spectral power distribution of a first wavelength range of 180-220 nm, especially 200-220 nm, relative to the spectral power distribution in the wavelength range of 180-780 nm, especially 180-400 nm, may be higher during at least part of the microbic lighting mode than during at least part of the standard lighting mode, especially at least 30% higher, such as at least 60% higher, especially at least 100% higher.

Similarly, in further embodiments, a relative spectral power distribution of a first wavelength range of 222 nm+/−5 nm, relative to the spectral power distribution in the wavelength range of 200-780 nm, especially 200-400 nm, may be higher during at least part of the microbic lighting mode than during at least part of the standard lighting mode, especially at least 30% higher, such as at least 60% higher, especially at least 100% higher.

In further embodiments, a relative spectral power distribution of a first wavelength range of 270 nm+/−10 nm, relative to the spectral power distribution in the wavelength range of 200-780 nm, especially 200-400 nm, may be higher during at least part of the microbic lighting mode than during at least part of the standard lighting mode, especially at least 30% higher, such as at least 60% higher, especially at least 100% higher.

In further embodiments, a relative spectral power distribution of a first wavelength range of 405 nm+/−5 nm, relative to the spectral power distribution in the wavelength range of 200-780 nm, especially 200-750 nm, may be higher during at least part of the microbic lighting mode than during at least part of the standard lighting mode, especially at least 30% higher, such as at least 60% higher, especially at least 100% higher.

In further embodiments, a relative spectral power distribution of a first wavelength range of 460 nm+/−10 nm, relative to the spectral power distribution in the wavelength range of 200-780 nm, especially 200-750 nm, may be higher during at least part of the microbic lighting mode than during at least part of the standard lighting mode, especially at least 30% higher, such as at least 60% higher, especially at least 100% higher.

Similarly, in further embodiments, a relative spectral power distribution of a first wavelength range of 460 nm+/−5 nm, relative to the spectral power distribution in the wavelength range of 180-780 nm, especially 200-750 nm, may be higher during at least part of the microbic lighting mode than during at least part of the standard lighting mode, especially at least 30% higher, such as at least 60% higher, especially at least 100% higher.

In embodiments, the first wavelength range may have a central wavelength λ_{c} and a spread λ_{S}, such that the first wavelength range comprises the wavelength range of λ_{c}−λ_{S}−λ_{c}+λ_{S}. In particular, in such embodiments, a relative spectral power distribution of the first wavelength range, relative to the spectral power distribution in the wavelength range of 180-780 nm, such as in the wavelength range of 200-750 nm, may be higher during at least part of the microbic lighting mode than during at least part of the standard lighting mode, especially at least 30% higher, such as at least 60% higher, especially at least 100% higher. In embodiments, the spread λ_{S} may be ≤10 nm, such as ≤5 nm, especially ≤2.5 nm, such as ≤1 nm. In further embodiments, the spread λ_{S} may be ≥0.2 nm, such as ≥0.5 nm, especially ≥1 nm. For instance, in embodiments 0.2 nm≤λ_{S}≤10 nm, such as 1 nm≤λ_{S}≤5 nm. In further embodiments, central wavelength λ_{c} may be selected from the group comprising 210 m, 222 nm, 230 nm, 254 nm, 270 nm, 405 nm, and 460 nm.

Hence, in embodiments, λ_{c} may be 210 nm, especially wherein λ_{S}≤10 nm, such as ≤5 nm, especially ≤2.5 nm. In further embodiments, λ_{c} may be 222 nm, and λ_{S} may be ≤5 nm, such as 5 nm. In further embodiments, λ_{c} may be 230 nm, especially wherein λ_{S}≤10 nm, such as ≤5 nm, especially ≤2.5 nm. In further embodiments, λ_{c} may be 254 nm, especially wherein λ_{S}≤10 nm, such as ≤5 nm, especially ≤2.5 nm. In further embodiments, λ_{c} may be 270 nm, and λ_{S} may be ≤10 nm, such as 10 nm. In further embodiments, λ_{c} may be 405 nm, and λ_{S} may be ≤5 nm, such as 5 nm. In further embodiments, λ_{c} may be 405 nm, and λ_{S} may be ≤10 nm, especially ≤5 nm, such as 5 nm. Hence, in embodiments, a relative spectral power distribution of a first wavelength range of 405 nm+/−5 nm, relative to the spectral power distribution in the wavelength range of 200-780 nm, especially 380-780 nm, such as 380-750 nm, may be higher during at least part of the microbic lighting mode than during at least part of the standard lighting mode, especially at least 30% higher, such as at least 60% higher, especially at least 100% higher.

Some wavelengths and wavelength ranges may be particularly suitable for promoting the persistence of the first microbes, especially with respect to the second microbes. In particular, these wavelengths or wavelength ranges may (substantially) differentially affect different microbes (also see below).

Hence, in further embodiments, the microbic lighting mode may comprise providing first device radiation having a wavelength selected from the range of <240 nm, such as from the range of 100-240 nm, especially from the range of 180-240 nm. In further embodiments, the microbic lighting mode may comprise providing first device radiation having a wavelength selected from the range of 250-280 nm, especially from the range of 260-280 nm. In particular, the relative spectral sensitivity of viruses and bacteria may often strongly depend on the UV wavelength. The differences in relative spectral sensitivity between the different microbes at a given wavelength may generally be most pronounced in the 260 nm-280 nm range and for UV wavelengths below 240 nm, wherein the spectral sensitivity delta between the microbes may continue to increase with lower UV wavelengths.

A desired differential effect of lighting on different microbes, especially between the first microbes and the second microbes, may be obtained at a specific wavelength. Hence, in further embodiments, the microbic lighting mode may comprise providing first device radiation having a (narrow) peak wavelength λ_{P}, especially wherein at least 30% of the spectral power in the wavelength range of λ_{P}−5−λ_{P}+5 nm falls within a peak wavelength range, such as at least 50%, especially at least 70%. In further embodiments, the peak wavelength range may especially comprise the range of λ_{P}−1−λ_{P}+1 nm, such as the range of λ_{P}−0.5−λ_{P}+0.5 nm. For instance, in embodiments, 70% of the spectral power of the first device radiation in the wavelength range of 400-410 nm may fall in the range of 404-406 nm, especially in the range of 404.5-405.5 nm.

In further embodiments, the control system may be configured to control the microbic lighting mode, especially the light generating device, in dependence of the related input signal, especially in dependence of the (received/sensed) microbiome influencing parameter. For instance, in embodiments, the control system may be configured to control the light generating device to provide first device radiation if the microbiome influencing parameter exceeds a threshold value. In further embodiments, the control system may be configured to control the light generating device to provide first device radiation upon determining based on the microbiome influencing parameter that an (inferred/predicted) abundance of first microbes will fall below a threshold value, or, similarly, that an (inferred/predicted) abundance of second microbes will exceed a threshold value.

Hence, the control system may be configured to steer the microbiome (composition) by controlling the light generating device, especially in dependence of the microbiome-influencing parameter. For instance, the microbiome-influencing parameter may suggest (or imply) a (to-be) increased prevalence of undesired second microbes, and the control system may control the light generating device to promote the persistence of the first microbes with respect to the second microbes. Similarly, the microbiome-influencing parameter may suggest (or imply) a presence of desired first microbes, and the control system may control the light generating device to promote the persistence of the first microbes with respect to second microbes, other than the first microbes, thereby facilitating the first microbes to settle (in a persistent manner) in the animal residence.

In further embodiments, the microbiome influencing parameter may be selected from one or more of (a) a presence of the first microbes and (b) a presence of the second microbes in the animal residence.

In further embodiments the microbiome influencing parameter is selected from the group of temperature, relative humidity, moisture level of the floor surface, light from a source other than the light generating device, ventilation, and ground cover material in the animal residence. For instance, modern dairy cow barns in Europe may typically have large-sized openings to the outside world to let fresh air in. The windows are opened more in the summer than in the winter, in view of temperature, resulting in more introduction of (“good”) first microbes from the outside air during the summer time than during the winter. Similarly, dairy cows in the Netherlands may typically spend some time on the pasture during the summer but may remain inside the barn during (most of) the winter. Hence, the cows may be exposed to a varied microbiome during the summer, but may be exposed to a limited indoor microbiome during winter. Hence, the spectral power distribution of the first device radiation may be selected in view of (outside) temperature. For instance, in embodiments, the lighting system may be (only) used during the wintertime to enrich the microbiome inside the stable.

The term “ventilation” may herein refer both to natural ventilation, such as via openings in wall(s) of the animal residence, and to ventilation provided by a ventilation system.

Hence, in further embodiments, the microbiome-influencing parameter may be selected from the group of season of the year, indoor or outdoor time of the animal, and time of the day.

In further embodiments, the microbiome influencing parameter may be selected from the group of type of animal, rearing stage of the animal(s), condition of the animal(s), feed for the animal(s), including type of feed and feeding time, spatial density of the animal(s), activity of the animal(s), such as movement, urination and defecation, cleansing of the animal residence, such as ongoing cleansing, or such as recent cleansing, treatment of the animal(s), including type of treatment, such as an antibiotic treatment, and treatment time, (expected) addition/exchange of the animal(s), (expected) human activity in the animal residence, stress level of the animal, and fear behavior of the animal. For instance, the gut microbiota of pigs housed in natural outdoor environments may typically be dominated by Firmicutes, in particular by Lactobacillus spp., whereas pigs housed under (excessively) hygienic conditions in indoor environments may display a reduced number of lactobacilli and higher numbers of Bacteroidetes and Proteobacteria including potentially pathogenic phylotypes. This illustrates that the pig's development in environments of excessive hygiene may hinder the natural progression towards an adult-type balanced gut microbiota, despite the acquisition of a highly diverse microbiota in early life, for instance at birth. In particular, the Firmicutes may be reduced in isolator-reared animals when compared to outdoor-reared littermates. In particular, early life may be a crucial developmental period during which continual exposure to environmental microbes is required to drive the ‘stabilization’ of the gut microbiota towards a desired adult phenotype. Similarly, the construction form of a rearing environment may significantly shape the microbial community of growing pigs. For instance, the flooring and bedding system and the bacteria present on surfaces of the animal residence may directly impact on the developmental dynamic of microbial communities of pigs. Similarly, also for horses the animal rearing conditions in an animal residence may change over time. For instance, horses may in a first time period typically be kept on wood shavings while in a second time period they may be kept on straw. The horses may be fed haylage or dry hay. Further, an antibiotic treatment may disrupt the (gut) microbiota of the animal(s). In particular, it may be beneficial to provide a bacterial reservoir of first microbes to facilitate fast re-colonization of the animal. Hence, in embodiments, after animals have been treated with antibiotics, the first device radiation may be used to increase the concentration of first microbes in the (rearing) environment of the animal.

For instance, after dispensing Bacillus ferments (typically found in soil and water), a previously cleaned building surface may be colonized within 45 minutes.

As indicated above, in embodiments, the microbiome influencing parameter may comprise an (expected) addition/exchange of the animal(s). In this case, the system of the invention may proactively take action in anticipation of (farm) animals to be added/removed in the animal residence. For instance, the expectation is that a pregnant cow will result in a calf. Hence, the system may proactively prepare the microbiome to be maximally suited for the calf before the calf is born. Similarly, if prolonged human activity in the stable is expected, for instance maintenance work inside the stable or hoof trimming of a cow, the microbiome in the animal residence may be made ready to improve the microbiome in the working environment for the human worker, and/or to account for any effects of the human and/or the work on the microbiome.

In embodiments, the system may comprise one or more actuators to further control the microbiome management, such as by controlling a microbiome influencing parameter. For instance, the control system may be configured to control one or more of temperature, humidity, air flow, number of animals in the animal residence, etc. etc. (see examples of the microbiome influencing parameters above). Hence, in addition to controlling the (first) device light, also (other) microbiome influencing parameters may be controlled (in dependence of a sensed microbiome influencing parameter. This control may e.g. be based on one or more of a feedback or feed forward control.

The microbiome may impact the fear response of an animal. In particular, germ-free animal models may have markedly different fear responses as compared to conventionally colonized animals. Hence, an unusual fear response may be indicative of a microbial unbalance. In particular, individual differences in composition of the microbiome may impact fear behavior acquisition and expression. Hence, in specific embodiments, the microbiome influencing parameter may comprise fear behavior of the animal(s). Thereby, the fear response of the animal may be modulated, especially improved, which may provide an overall improved wellbeing of the (farm) animal. Similarly, an unbalanced microbiota may give rise to stress and/or stress may lead to changes in the microbiota. Hence, stress may be indicative of a microbiome unbalance.

Hence, in further embodiments, the control system may be configured to control the light generating device in dependence of information from an animal rearing management system, i.e., the system may comprise or be functionally coupled to an animal rearing management system, wherein the control system is configured to receive and/or retrieve information from the animal rearing system, and wherein the control system is configured to control the light generating device in dependence on the information from the animal rearing management system.

In embodiments, the control system may be configured to control one or more of the spectral power distribution of the first device radiation, a duty cycle of the first device radiation, a dynamic lighting effect of the first device radiation, a spatial direction of (a first beam of) the first device radiation, and the intensity of the first device radiation in dependence of the related input signal. Hence, the control system may be configured to control one or more properties of the first device radiation in dependence of the related input signal. The control system may especially be configured to control the spectral power distribution of the first device radiation in dependence of the related input signal.

In embodiments, the system, especially the control system, may have an operational mode, especially a microbic application mode (see below), and/or especially a microbic lighting mode. The term “operational mode” may also be indicated as “controlling mode”. The system, or apparatus, or device (see further also below) may execute an action in a “mode” or “operational mode” or “mode of operation”. Likewise, in a method an action, stage, or step may be executed in a “mode” or “operation mode” or “mode of operation”. This does not exclude that the system, or apparatus, or device may also be adapted for providing another operational mode, or a plurality of other operational modes. Likewise, this does not exclude that before executing the mode and/or after executing the mode one or more other modes may be executed. However, in embodiments a control system (see further also below) may be available, that is adapted to provide at least the operational mode. Would other modes be available, the choice of such modes may especially be executed via a user interface, though other options, like executing a mode in dependence of a sensor signal or a (time) scheme, may also be possible. The operational mode may in embodiments also refer to a system, or apparatus, or device, that can only operate in a single operation mode (i.e. “on”, without further tunability).

The system may comprise a microbe (mist) dispenser device. The microbe dispenser device may especially be configured to provide an emission of first microbes, i.e., the microbe dispenser device may be configured to continuously, periodically, or intermittently provide first microbes to (an indoor space of) the animal residence. In embodiments, the microbe dispenser device may especially be configured to provide in the microbic application mode a spray of the first microbes. In further embodiments, the microbe dispenser device may especially be configured to provide in the microbic application mode a (dry) powder of the first microbes, i.e., to provide the first microbes in a powder form.

In embodiments, a microbe dispenser may employ ultrasonic to create aerosols with first microbial loading.

The system of the invention may thus both provide (beneficial) first microbes and provide first device radiation suitable for promoting the persistence of the first microbes relative to second microbes. In particular, the first device radiation may be beneficial for the growth of the first microbes, and/or may be detrimental to the growth of the second microbes. Thereby, the first device radiation may provide the first microbes a competitive advantage over the second microbes, allowing the first microbes to settle and persist in the animal residence.

In particular, the system of the invention may actively enhance microbial diversity throughout an animal residence, including by deliberate introduction of wanted microbial species, such as those commonly found in nature on plants and leaves. These microbes may further mitigate the side effects of (overzealous) disinfection of the animal residence, for instance with light-based disinfection or ionizers, and may provide competition for harmful microbes arriving later.

Hence, the invention may relate to both dispensing (or “applying”) microbes and further promoting the wanted microbiome in the room with the help of tailored lighting recipes. In embodiments, the microbe dispensers may be actuated depending on a current context of the animal residence, as well as depending on the past/current/predicted operation status of a disinfection system and an (expected) occupancy status or (expected) activity in the residence.

In particular, in embodiments, the microbe dispenser device may (be configured to) have a microbe emission region (or range), i.e., the microbe dispenser device may (be configured to) provide the first microbes to a microbe emission region. In embodiments, the microbe emission region may be a 2D region, such as a surface. In further embodiments, the microbe emission region may be a 3D region, such as an (indoor) space, especially a rearing space.

Especially, in embodiments, the microbe dispenser device may be configured to provide in a microbic application mode an emission of first microbes and essentially no second microbes, i.e., the emission is (essentially) devoid of second microbes.

As partly mentioned, the system, especially the control system, may have a microbic application mode. In the microbic application mode, the microbe dispenser device may (be configured to) provide a (microbe) emission of the first microbes. In particular, in the microbic application mode, the microbe dispenser device may (be configured to) provide the emission to the microbe emission region. Hence, during the microbic emission mode, the microbe emission region may be populated with the first microbes.

In particular, the system, especially the light generating device, may be configured to provide the first device radiation to the dispensed first microbes, i.e., to (at least part of) the microbe emission region. In particular, in embodiments, the light generating device may be configured to provide a first beam of first device radiation, wherein the microbe emission region and the first beam may at least partly spatially overlap. For instance, the microbe emission region may comprise a surface and the first beam may illuminate at least part of the surface, or the microbe emission region may comprise a (3D) space and the first beam may pass through at least part of the space. Hence, in embodiments, the microbe emission region and the first beam may (at least partially) spatially overlap at a distance from the microbe emission device and/or the light generating device, especially from the microbe emission device, or especially from the light generating device, such as at a distance selected from the range of 0.5-10 m.

In particular, the first beam may be divergent (with respect to the light generating device), and may thus cover a large area at a larger distance from the light generating device. Hence, in embodiments, the microbe emission region and the first beam may have a shared region, i.e., they overlap in the shared region, wherein the shared region, such as a shared volume, or such as a shared surface, may have a frustum-shape.

In further embodiments, the light generating device may be configured to (during the microbic lighting mode) provide the first device radiation, especially the first beam, to at least part of the microbe emission region.

The first beam and the microbe emission region may, in embodiments, spatially overlap for at least 30% of the microbe emission region, especially at least 50%, such as at least 70%, including 100%, especially with regards to an area of a surface, or especially with regards to a volume of a space.

In further embodiments, the emission of first microbes and the providing of the first beam may also temporally overlap (also see below).

In embodiments, the microbic lighting mode and the microbic application mode may temporally overlap, i.e., the microbe dispenser device may provide an emission of first microbes to the microbe emission region while the light generating device provides the first beam of first device radiation.

In further embodiments, the microbic lighting mode be temporally arranged after the microbic application mode. Hence, the microbe dispenser device may (first) provide the first microbes to the microbe emission region, and the light generating device may subsequently provide the first beam of the first device radiation, especially to at least part of the microbe emission region. Such embodiments may be particularly beneficial when the first device radiation has a spectral power distribution selected for promoting the growth of the first microbes.

Hence, in embodiments, the microbic lighting mode may overlap in time with the microbic application mode or may be subsequent in time to the microbic application mode.

The system, especially the control system may further have a disinfection mode. In the disinfection mode, the light generating device may (be configured to) provide disinfection radiation. In embodiments, the disinfection radiation may comprise UV radiation. In further embodiments, the disinfection radiation may comprise visible near UV radiation, especially comprising a disinfection wavelength selected from the range of 380-450 nm, such as from the range of 400-410 nm, especially of (about) 405 nm. In further embodiments, the disinfection radiation may comprise IR radiation, especially comprising an IR wavelength selected from the range of 750-2000 nm, such as from the range of 750-1500 nm. In further embodiments, the IR wavelength may be ≤900 nm, such as selected from the range of 750-900 nm. In yet further embodiments, the IR wavelength may be ≥780 nm, even more especially ≥900 nm, such as selected from the range of 900-2000 nm, especially from the range of 900-1500 nm, such as from the range of 900-1100 nm. In particular, in the disinfection mode, the light generating device may (be configured to) provide a disinfection beam of disinfection radiation, wherein the disinfection beam at least partially overlaps with the microbe emission region. Hence, in embodiments, in the disinfection mode, the light generating device may (be configured to) provide disinfection radiation to the microbe emission region.

In further embodiments, the system, especially the light generating device, may be configured to provide in a disinfection mode (of the system) one or more of (a) disinfection radiation and (b) charged particles, especially disinfection radiation, or especially charged particles. Especially, the disinfection radiation may comprise one or more of (i) UV radiation having one or more wavelengths selected from the wavelength range of 100-380 nm, (ii) visible near UV radiation having one or more wavelengths selected from the wavelength range of 380-495 nm, and (iii) IR radiation having one or more wavelengths selected from the wavelength range of 750-950 nm.

Hence, in further embodiments, the system may comprise an ionizer device, wherein the ionizer device is configured to provide the charged particles (during the disinfection mode).

The term visible near UV radiation may herein refer to (disinfection) radiation in the visible spectrum but close to the UV spectrum. In particular, in embodiments, the visible near UV radiation may comprise the wavelength range of 380-495 nm, such as the wavelength range of 380-450 nm, especially the wavelength range of 380-420 nm.

In embodiments, the control system may be configured to control the light generating device. In further embodiments, the control system may be configured to control the microbe dispenser device. In particular, the control system may be configured to control one or more of the first device radiation, the disinfection radiation, and the emission of the first microbes, especially in dependence of one or more of the related input signal, especially the related sensor signal, a timer, a user interface, and a predetermined program.

For instance, in embodiments, the control system may be configured to control the one or more of the first device radiation, the disinfection radiation, and the emission of the first microbes based on the related input signal of the input system, especially based on the related sensor signal from a sensor, such as a sensor selected from the group comprising a movement sensor, a presence sensor, an activity detection sensor, a people counting sensor, a distance sensor, an ion sensor, a gas sensor, a volatile organic compound sensor, a pathogen sensor, an airflow sensor, a sound sensor, a temperature sensor, and a humidity sensor. For example, in embodiments, the sensor may comprise a pathogen sensor, and the sensor signal may indicate a high level of an (undesirable) second microbe, which may cause the control system to initiate one or more of the first device radiation, the disinfection radiation, and the emission of the first microbes, especially the disinfection radiation. In further embodiments, the sensor may comprise a temperature sensor, and the sensor signal may indicate that the temperature is rising, which may provide a competitive benefit to the first microbes or, alternatively, to (part of) the second microbes. Hence, the control system may be configured to respond to a changing temperature by, for instance, increasing the emission of first microbes and/or increasing the first device radiation. In further embodiments, the sensor may comprise an activity sensor configured to detect (an) activity of an animal (or a human) in the animal residence, such as pooing, peeing, sleeping, feeding, body posture, interaction between animals (e.g. fighting), and an eating activity. For instance, fighting between animals may result in bodily fluids, such as blood, being deposited on surfaces of the animal residence, such as on the floor. In such embodiments, the control system may, for example, increase the microbe emission after the detected activity, may modulate, especially stop, disinfection radiation during the activity, and may increase wavelengths in the first device radiation that contribute to vitamin D generation (see below). Further, if the system further comprises a pathogen sensor, the system may be configured to determine a pathogen status in the indoor space after the activity to re-assess the need for subsequent disinfection. In further embodiments, the sensor may comprise a presence sensor, and the sensor signal may also indicate that an animal, especially a human, has entered the room, which may cause the control system to modulate the disinfection radiation, especially stop the disinfection radiation, or especially alter the wavelength of the disinfection radiation, as UV radiation may also be harmful to animals, such as humans. For instance, the table below may indicate properties of different disinfection wavelength ranges, including disinfection efficiency for different types of micro-organisms, and safety (to humans):

| | | | (Relative) | | | | |
|---|---|---|---|---|---|---|---|
| | | | sterilization | | | | |
| | Short | Wavelength | effectiveness | | Safe | Vitamin D | Ozone |
| Name | name | (nm) | Bacteria | Viruses | Radiation | generation | generation |
| Near | NIR | 750-950 | + | +/− | +++ | | |
| Infrared | | | | | | | |
| Violet | V | 380-420 | +/− | − | + | | |
| Ultra- | UV-A | 315-380 | + | − | + | | |
| violet A | | | | | | | |
| Ultra- | UV-B | 280-315 | + | +/− | +/− | + | |
| violet B | | | | | | | |
| Near ultra- | Near | 230-280 | ++ | ++ | − | | |
| violet C | UV-C | | | | | | |
| Far ultra- | Far | 190-230 | +++ | +++ | + | | +/− |
| violet C | UV-C | | | | | | |
| Extreme | Extreme | 100-190 | +++ | +++ | − | | + |
| ultra- | UV-C | | | | | | |
| violet C | | | | | | | |

The use of UV for disinfection may be well-established. Also IR light may also have a disinfectant effect. In general, the term infrared refers to wavelengths above 750 nm, of which near infrared (NIR) describes wavelengths 750-1400 nm, of which the range of 750-950 nm may be particularly suitable for IR disinfection. An advantage of using IR light as disinfection light is that it may work better than UV light on certain surfaces and for certain microbes. For example, it may be well-known that NIR disinfection (750-950 nm) achieves an 80% to 99.9% or 2-3 log reduction of iron-dependent and some other types of bacteria and fungi. Furthermore, the absorption spectrum of each microbe may show some distinct absorption peaks. Consequently, specific IR wavelengths can be selected so as to ‘excite’ specific bond types in the specific molecules in, for instance, targeted second microbes.

Moreover, in practical disinfection lighting conditions, the target surface may be partially blocked. While a certain material may be transparent at IR frequencies (e.g. thin plastic), it may not be transparent for 405 nm or UV disinfection light. It is known that infrared light can pass through many materials which visible or UV light cannot pass through. However, the reverse is also true. There are some materials such as glass which can pass visible 405 nm light but not infrared.

For instance, Far Infrared rays can penetrate into the human body tissue up to 1.5 to 2.8 inches while UV light may generally be absorbed in the outer dead skin layer. Similarly, if a sheet of paper lies on a desk surface, IR may be scattered by the paper, but some of the IR light may nevertheless pass through the paper and reach the surface underneath.

In addition, additional synergetic effects between IR and UV disinfection light sources, or other dual-functions of either the IR or UV, may be realized. For instance, the IR light source may further be used to provide warmth, according to a different setting, for heating up the floor of the animal residence. The IR light also may be used to provide wellness feeling by heating bodies in a room directly without heating up the air. Absorbing the heat into the skin, which feels like warm sun rays on a spring day may be particularly relaxing and incredibly comforting for the animals.

As an additional advantage, the IR light may be used to prevent mold or mildew. In many spaces in wet rooms, silicone sealing may get attacked by mildew and may require special treatment. The IR light may be used to heat up and dry the walls (instead of the air like conventional heaters) and hence provide a mold prevention function in times of high humidity.

It appears that the combination of UV and IR light sources may create a synergistic effect for killing pathogens. However, IR disinfection may also have drawbacks: if a high IR dose is administered to an object, it may heat to a high temperature, which may damage the object. It may hence be advantageous that a to-be-disinfected surface is covered by water film when applying the IR disinfection.

In further embodiments, the control system may be configured to control the one or more of the first device radiation, the disinfection radiation, and the emission of the first microbes based on the timer. For instance, the control system may be configured to (have the microbe dispenser device) provide a microbe emission at least every hour. Hence, if an hour has passed since a last (scheduled or spontaneous) microbe emission, the control system may (have the microbe dispenser device) provide a microbe emission of first microbes.

In further embodiments, the control system may be configured to control the one or more of the first device radiation, the disinfection radiation, and the emission of the first microbes based on the user interface. In particular, the control system may comprise the user interface. A user may, for instance, provide input to the control system to initiate or cease an operation.

In further developments, the control system may be configured to control the one or more of the first device radiation, the disinfection radiation, and the emission of the first microbes based on the predetermined program. For instance, the control system may be configured to cultivate the microbiome such that a desired microbiome is present at the (feeding) troughs at feeding time. Hence, in embodiments, the control system may be configured to employ the one or more of the first device radiation, the disinfection radiation, and the emission of the first microbes during (at least part of) the night for indoor microbiome management in the animal residence. In embodiments, the control system may be configured to dynamically adapt the predetermined program based on a schedule, such as based on a calendar. In further embodiments, the control system may be configured to predict activities in the indoor space, such as based on historic data related to past activities, and adapt the predetermined program accordingly.

In further embodiments, the control system may have access to an external database, especially wherein the external database comprises information on the first microbes and/or the second microbes. In particular, the control system may be configured to retrieve information on the first microbes and/or the second microbes from the external database, especially wherein the information comprises data on spectral sensitivities of the first microbes and/or the second microbes. In embodiments, the control system may be configured to retrieve information on the (geographic) prevalence of second microbes from the external database, such as based on a GPS location of the animal residence, especially also taking into account a wind direction or the surroundings of the animal residence (forest, pasture fields, thickly settled area with many building structure and streets), such as when the animal residence is open to the outdoors. Hence, the control system may be configured to determine the (likely) presence of second microbes based on information retrieved from the external database, and may select the spectral power distribution of the first device radiation based on the presence of the second microbes.

Similarly, in embodiments, the control system may comprise or be functionally coupled to a user interface. In particular, in embodiments, the input system may comprise the user interface. The user interface may be configured for receiving input and providing the input to the control system. In embodiments, the user interface may be configured for receiving input related to the spectral sensitivities of the first microbes and/or the second microbes. In further embodiments, the user interface may be configured for receiving input related to the type of second microbes present in the indoor space. In further embodiments, the user interface may be configured for receiving input related to the microbiome influencing parameter.

The disinfection radiation may also impair the first microbes, i.e., the first microbes may also be vulnerable to the disinfection radiation. Hence, in embodiments, the control system may be configured to control a microbe emission rate of the (emission of the) first microbes in dependence of the disinfection radiation.

In particular, in further embodiments, the microbic application mode and the disinfection mode may at least partly overlap in time, especially wherein relative to a baseline emission rate, such as a daily average microbe emission rate, the microbe emission rate during at least part of the disinfection mode may be higher than the baseline emission rate. Hence, as the first microbes are being inactivated/killed due to the disinfection radiation, the microbe dispenser device may simultaneously dispense the first microbes to maintain a desired balance. In particular, during periods when the disinfection radiation is actuated, extra first microbes may be actively dispensed into the indoor space to compensate for the effect of the UV light unintentionally deactivating some of the wanted biome on the surfaces. Hence, the microbe dispenser device may concurrently replenish those good bacteria, which got unintentionally deactivated by the disinfection radiation.

In embodiments, the control system may be configured to control the light generating device in dependence of the disinfection mode. In further embodiments, the control system may be configured to control the microbe dispenser device in dependence of the disinfection mode.

In further embodiments, the microbic application mode and the disinfection mode may be temporally separated, i.e., the microbic application mode and the disinfection mode do not overlap in time. In such embodiments, relative to the baseline microbe emission rate, the microbe emission rate after the disinfection mode may be higher than the baseline microbe emission rate. Hence, after the disinfection radiation has disinfected a space/region, especially the microbe emission region, the space/region may be repopulated with the first microbes.

Hence, the disinfection mode may especially be used to cleanse (or “prepare”) the microbe emission region for the first microbes. In particular, the disinfection radiation may be used to remove (or “deactivate” or “kill”) second microbes or viruses inhabiting (at least part of) the microbe emission region, thereby reducing the competition for the first microbes in the microbe emission region. Hence, the disinfection mode may facilitate the first microbes persisting on the microbe emission region. In further embodiments, the disinfection mode may (thus) be temporally arranged (directly) before the microbic application mode.

The term “baseline emission rate” may herein refer to a default microbe emission rate. In embodiments, the microbe emission rate may be (essentially) constant over time. In further embodiments, the microbe emission rate may follow a temporal pattern. In further embodiments, the microbe emission rate may be irregular over time. In further embodiments, the baseline emission rate may be (essentially) 0, and the microbic application mode may be initiated (only) when specific conditions apply, such as when user input is received, and/or such as (directly) after the disinfection mode.

In further embodiments, the baseline emission rate may be a daily average emission rate.

In further embodiments, the control system may be configured to select the spectral power distribution of the first device radiation to (a) promote the persistence, especially growth, of the first microbes (relative to the second microbes), and (b) to deactivate a virus.

The control system may have access to a (predetermined) target microbiome composition. In particular, the control system may be configured to control the light generating device in order to steer an actual microbiome composition in the animal residence to the (predetermined) target microbiome composition. The term “target microbiome composition” may generally comprise a range of target microbiome compositions, i.e., there may be some freedom with regards to specific microbes and/or with regards to relative prevalence of such microbes. It may, however, be impossible to achieve a desired target microbiome composition within a given timeframe, such as if a microbiome influencing parameter cannot be fully counteracted, or such as if a desired first microbe is not present in the animal residence and is not available for dispensing via the microbe dispenser device. In such scenarios, the control system may yet control the light generating device to steer towards the desired target microbiome composition, such as based on a scoring function. In particular, the control system may be configured to execute an optimization algorithm to select a spectral power distribution of the first device radiation to steer the microbiome composition towards the target microbiome composition (in view of any restraints).

Hence, in embodiments, the control system may have access to a predefined target microbiome composition for (at least part of) the animal residence, and the control system may especially be configured to control the light generating device in dependence of the microbiome influencing parameter and the target microbiome composition.

In particular, for example, the microbiome influencing parameter may push an actual microbiome composition to the target microbiome composition, and the control system may optionally finetune microbiome composition by controlling the light generating device. However, in a further example, the microbiome influencing parameter may push the actual microbiome composition away from a target microbiome composition, such as via an increasing prevalence of second microbes, or such as via a reduction in (specific) first microbes, and the control system may control the light generating device to counteract the microbiome influencing parameter.

Hence, in embodiments, the control system may (be configured to) determine a microbiome effect of the microbiome influencing parameter. In further embodiments, the control system may (be configured to) select a lighting intervention based on the microbiome effect, and especially to control the light generating device based on the lighting intervention. In particular, the control system may control the spectral power distribution of the first device radiation based on the lighting intervention.

As alluded to above, the control system may infer (changes to) the microbiome (composition) based on the microbiome influencing parameter, as well as based on historical data. It may, however, be particularly insightful if the control system has access to data on (at least part of) an actual microbiome composition in the animal residence. Hence, in embodiments, the sensor may comprise a microbiome sensor, wherein the microbiome sensor is configured to determine a microbiome-related parameter and to provide a related microbiome signal to the control system, wherein the control system is configured to determine (at least part of) a current microbiome composition based on the related microbiome signal.

For instance, in further embodiments, the sensor may comprise one or more of a 16S-RNA sequencer or an 18S-RNA sequencer.

In embodiments, the microbe dispenser device may comprise a cartridge holder. The cartridge holder may especially be configured to detachably host one or more cartridges, especially a plurality of cartridges. In embodiments, the cartridge holder may be configured to host at least a cartridge comprising (at least part of) the first microbes. In further embodiments, the cartridge holder may be configured to host a plurality of cartridges comprising (different) first microbes. In further embodiments, the cartridge holder may be configured to host a cartridge comprising a type of odor, especially an odor compound.

In specific embodiments, the cartridge holder may be configured to host a plurality of cartridges, wherein two or more of the plurality of cartridges are filled with material differing in types of first microbes.|

In further embodiments, the system may comprise a cartridge sensor configured to determine a remaining capacity (or volume) of first microbes in cartridges in the microbe dispenser device.

Hence, the microbe dispenser device may comprise (cartridges comprising) different types of first microbes. In embodiments, the control system may be configured to control the microbe dispenser device to emit part of the (part of) the first microbes based on a current (determined/estimated) microbiome in the animal residence and the currently allowable spectral power distributions in the animal residence.

Hence, the control system may further select the spectral power distribution in view of the animals in the (indoor space of the) animal residence. For example, animals may have different light sensitivities during different stages of development, and specific wavelengths may be preferably avoided for, for example, young animals. For example, the retina of young animals may be more sensitive to UV radiation as compared to the retina of older animals.

In further embodiments, the control system may be configured to control the light generating device in dependence of the microbiome influencing parameter, the current microbiome composition, the current availability of first microbes in the microbe dispenser cartridge and the target microbiome composition. Hence, the control system may take into account expected changes and possibilities for (actively) introducing first microbes into the animal residence, for selecting a suitable spectral power distribution for steering the microbiome composition in the animal residence towards the target microbiome composition.

In further embodiments, the microbe dispenser device may comprise a mist dispenser, especially wherein the mist dispenser is configured to dispense the first microbes.

In a further aspect, the invention may provide a (lighting) device comprising the lighting system according to the invention. In embodiments, the device may comprise a housing. The housing may especially enclose at least part of the light generating device and, in further embodiments, at least part of the microbe dispenser device. In embodiments, the housing may essentially enclose the light generating device. In further embodiments, the housing may essentially enclose the microbe dispenser device.

In embodiments, the device may especially comprise a lighting device. In further embodiments, the lighting device may be selected from the group of a lamp, a luminaire, a projector device, a disinfection device, and an optical wireless communication device, especially a luminaire.

In further embodiments, the device may be configured such that a first beam of first device radiation has a first direction, and wherein a second beam of disinfection radiation has a second direction, wherein the first direction and the second direction have a mutual angle (α_{M}) selected from the range of 60-180°, especially from the range of 90-180°, such as from the range of 120-180°. In particular, in further embodiments, the device may be configured such that a first beam of first device radiation has a first direction parallel to a first optical axis (O1) (of the first beam); and wherein a second beam of disinfection radiation has a second direction parallel to a second optical axis (O2) (of the second beam), wherein the first direction and the second direction have a mutual angle (α_{M}) selected from the range of 60-180°, especially from the range of 90-180°, such as from the range of 120-180°. In particular, in such embodiments, the first device radiation and the disinfection radiation may be spatially separated. Especially, the microbe emission region may be spatially (essentially) non-overlapping with the disinfection radiation. Hence, the disinfection radiation may be provided to one part of the space, such as a ceiling, whereas the first device radiation may be provided to a different part of the space, such as to a table surface. Especially, the first direction may coincide with the first optical axis (of the first device radiation). Yet, in embodiments the second direction may coincide with the second optical axis (of the second beam of disinfection radiation).

In further embodiments, the device may especially comprise an upper air disinfection device configured to provide disinfection radiation to the ceiling and/or a (top) part of a wall.

In a further aspect, the invention may provide an animal residence system. The animal residence system may comprise an animal residence and the (lighting) system of the invention. In particular, the (lighting) system may be configured to control the indoor microbiome in the animal residence.

In embodiments, the animal residence may comprise a rearing space, especially wherein the light generating device of the lighting system is configured to provide the first device radiation to the rearing space.

In a further aspect, the invention may provide a method for indoor microbiome management in an animal residence. The method may comprise providing first device radiation to (an indoor space of) the animal residence, wherein a spectral power distribution of the first device radiation is selected for promoting persistence, especially growth, of first microbes relative to second microbes, other than the first microbes. The method may further comprise
receiving and/or sensing, especially receiving, or especially sensing, a microbiome influencing parameter and providing a related input signal. The method may further comprise controlling (a spectral power distribution of) the first device radiation in dependence of the related input signal.

In embodiments, the method may further comprise providing an emission of first microbes in (the indoor space of) the animal residence.

The method may especially comprise providing first device radiation and an emission of first microbes in the animal residence. In embodiments, a spectral power distribution of the first device radiation may be selected for promoting persistence, especially growth, of the first microbes relative to second microbes, other than the first microbes. In further embodiments, the spectral power distribution of the first device radiation may be selected for one or more of (i) promoting growth of the first microbes, (ii) deactivating second microbes, other than the first microbes, (iii) and deactivating second microbes more strongly than the first microbes.

Hence, in specific embodiments, the method may comprise providing in (an indoor space of) the animal residence (a) first device radiation, and (b) an emission of first microbes based on a microbiome influencing parameter; wherein a spectral power distribution of the first device radiation is selected for promoting persistence of the first microbes relative to second microbes, other than the first microbes.

In embodiments, the method may comprise a microbic lighting mode. The method, especially the microbic lighting mode, may comprise providing a first beam of first device radiation based on the microbiome influencing parameter.

In further embodiments, the method may comprise a microbic application mode. The method, especially the microbic application mode, may comprise providing an emission of the first microbes in a microbe emission region based on the microbiome influencing parameter.

In further embodiments, the microbic lighting mode and the microbic application mode may temporally overlap, i.e., the microbic lighting mode may overlap in time with the microbic application mode. In further embodiments, the microbic application mode may be temporally arranged (directly) after the microbic lighting mode, i.e., the microbic lighting mode may be subsequent in time to the microbic application mode.

In further embodiments, the first beam and the microbe emission region may at least partly spatially overlap.

The method may, in embodiments, further comprise a disinfection mode. In further embodiments, the method, especially the disinfection mode, may comprise directing at least part of disinfection radiation to an upper air space, such as to a ceiling, especially wherein the disinfection radiation comprises UV radiation. In further embodiments, the method, especially the microbic lighting mode, may comprise directing at least part of the first beam of first device radiation to a floor. In further embodiments, the method, especially the microbic application mode, may comprise directing at least part of the emission of the first microbes to the floor. In particular, in embodiments, the method may comprise providing disinfection radiation and microbic lighting radiation at a mutual angle (α_{M}) selected from the range of 60-180°, especially from the range of 90-180°, such as from the range of 120-180°. In specific embodiments, the method may comprise using lighting system or lighting device as described herein (and as claimed).

The lighting device or lighting system may be part of or may be applied in an animal residence, such as in a barn, a shed, a pen, a (farm)house, et cetera.

As e.g. bacillus first microbes can reduce odors, an odor VOC sensor may be useful as possible sensor device in the animal housing. Providing of e.g. bacillus microbes in an animal housing may in embodiments be based on the detected odor level. Therefore, providing microbes eventually remove at least part of the undesired smell from the animal housing. As indicated above, the sensor may comprise a volatile organic compound (VOC) sensor.

A temperature sensor may be useful for controlling when to dispense first microbes. For instance, bacillus-based product may substantially not work well below 5° C. and above 60° C. In this case, for instance a microbe dispenser, or other device to distribute first microbes may waits with applying the microbes until a temperature is in the temperature wherein the first microbe(s) is (are) active, and not provide first microbe(s) when the temperature is outside such temperature range. As indicated above, the sensor may comprise a temperature sensor.

The term white light (or “white radiation”) herein, is known to the person skilled in the art. It especially relates to light having a correlated color temperature (CCT) between about 2000 and 20000 K, especially 2700-20000 K, for general lighting especially in the range of about 2700 K and 6500 K, and for backlighting purposes especially in the range of about 7000 K and 20000 K, and especially within about 15 SDCM (standard deviation of color matching) from the BBL (black body locus), especially within about 10 SDCM from the BBL, even more especially within about 5 SDCM from the BBL.

### BRIEF DESCRIPTION OF THE DRAWINGS

Embodiments of the invention will now be described, by way of example only, with reference to the accompanying schematic drawings in which corresponding reference symbols indicate corresponding parts, and in which.

FIGS. 1A and 2A schematically depict embodiments of the system,

FIG. 1C schematically depicts a spectral power distribution for the standard lighting mode,

FIGS. 1B and 2B schematically depict embodiments of operational modes of the system, and

FIGS. 3A-B schematically depict relative spectral sensitivities S versus wavelength λ (in nm).”

The schematic drawings are not necessarily on scale. The schematic drawings are not necessarily on scale.

### DETAILED DESCRIPTION OF THE EMBODIMENTS

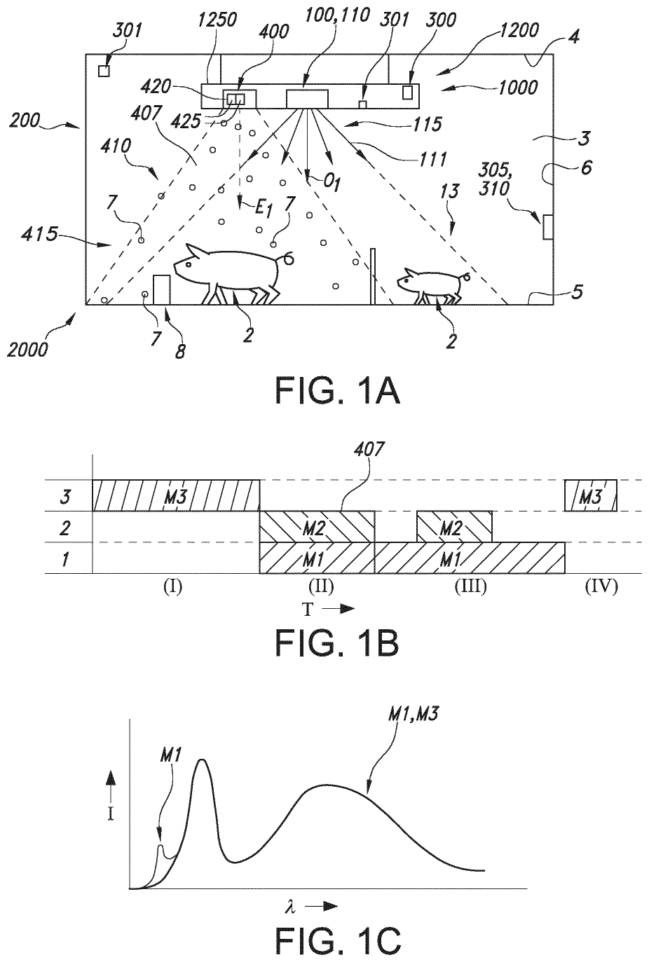

FIG. 1A schematically depicts an embodiment of the system 1000 for indoor microbiome management in an animal residence 200 of the invention. The system 1000 may especially comprise a lighting system. In the depicted embodiment, the system 1000 comprises a light generating device 100, a control system 300, and an input system 305, especially a sensor system 310. In embodiments, and especially in a microbic lighting mode, the light generating device 100 may be configured to generate first device radiation 111. In particular, a spectral power distribution of the first device radiation 111 may be selected for promoting persistence, especially growth, of the first microbes 7 relative to second microbes, other than the first microbes 7. In further embodiments, the input system 305 may be configured to receive and/or sense a microbiome influencing parameter and to provide a related input signal to the control system 300. Especially, the control system 300 may be configured to control the light generating device 100 in dependence of the related input signal.

Hence, the input system 305 may be configured to receive and/or sense a microbiome influencing parameter and to provide a related input signal to the control system, and the control system may be configured to control the light generating device 100 in dependence of the related input signal in order to manage a microbiome in the animal residence 200. For instance, the microbiome influencing parameter may suggest or imply an increasing prevalence of undesired second microbes, and the control system 300 may (control the light generating device 100 to) adapt a spectral power distribution of the first device radiation 111 in order to suppress (or “inactivate”) the second microbes. In particular, in embodiments, the control system may be configured to control the spectral power distribution of the first device radiation 111 for one or more of (i) promoting growth of the first microbes 7, (ii) deactivating second microbes, other than the first microbes 7, (iii) deactivating second microbes more strongly than the first microbes 7, and (iv) deactivating viruses, especially for one or more of (i)-(iii).

In further embodiments, the viruses may comprise first (desirable) viruses and second (undesirable) viruses, other than the first viruses. Hence, in embodiments, the method control system may be configured to control the spectral power distribution of the first device radiation 111 for deactivating second viruses more strongly than the first viruses. For instance, the first viruses may comprise bacteriophages configured to target the second microbes, whereas the second viruses may comprise bacteriophages configured to target the first microbes. The second viruses may further, for instance, comprise human and/or animal pathogens.

In further embodiments, the control system 300 may be configured to control one or more of the spectral power distribution of the first device radiation 111, a duty cycle of the first device radiation 111, a dynamic lighting effect of the first device radiation 111, a spatial direction of the first device radiation 111, especially of a first beam 115 of the first device radiation 111, and the intensity of the first device radiation 111 in dependence of the related input signal.

In further embodiments, the input system 305 comprises a sensor 310, wherein the sensor 310 is configured to sense the microbiome influencing parameter, and wherein the related input signal comprises a related sensor signal. Hence, the sensor 310 may be configured to sense the microbiome influencing parameter and to provide a related sensor signal to the control system 300.

In the depicted embodiment, the light generating device 100 may be configured to provide the first device radiation 111 to a floor 5 and a feeding element 8 of the animal residence 200. In further embodiments, the light generating device 100 may be configured to provide the first device radiation 111 to one or more of a ceiling 4 of the animal residence 200, a floor 5 of the animal residence 200, a wall 6 of the animal residence 200, a feeding element 8, such as a trough, in the animal residence 200, and a sleeping part 9, such as a straw bed, in the animal residence 200.

The current microbiome composition in the animal residence 200 may be informative with regards to (to-be expected) changes in the microbiome composition over time. For instance, if a specific first microbe 7 or second microbe is not present in the animal residence 200, it will not accumulate over time unless first introduced from externally. Further, microbes may synergize with specific (groups of) other microbes, such as by forming synergistic microbial communities. Hence, the presence of two or more particular species in the animal residence 200 may be indicative of a relatively enhanced persistence, especially growth, of one or both of the species. Similarly, microbes may compete for a same niche, which may result in a competitive exclusion. In such a case, the presence of a particular microbe may be indicative of a relatively reduced persistence for a different microbe. Hence, in embodiments, the microbiome influencing parameter may be selected from one or more of (a) a presence of first microbes 7, (b) a presence of second microbes and (c) a presence of viruses, especially first viruses and/or second viruses, in the animal residence 200, especially from one or more of (a) the presence of first microbes 7, and (b) the presence of second microbes in the animal residence 200.

Hence, in further embodiments, the sensor 310 may comprise a microbiome sensor 311, wherein the microbiome sensor 311 is configured to determine a microbiome-related parameter and to provide a related microbiome signal to the control system 300, especially wherein the control system 300 is configured to determine a current microbiome composition based on the related microbiome signal.

The composition of the microbiome in the animal residence 200, such as with regards to the first microbes 7, as well as with regards to the second microbes, may further be affected by a variety of factors, including animal behavior, temperature, humidity, antibiotic exposure, introduction of objects in the residence, food, et cetera. Hence, in embodiments, the microbiome influencing parameter may be selected from the group comprising temperature, relative humidity, light from a source other than the light generating device 100, ventilation, ground cover material in the animal residence 200, type of animal 2, rearing stage of the animals 2, condition of the animals 2, feed for the animals 2, treatment of the animals 2, season of the year, indoor or outdoor time of the animal 2, and time of the day.

In particular, the rearing stage of the animals 2 may both be a relevant microbiome influencing parameter, as well as a relevant parameter with regards to a desirable (target) microbiome for (an indoor space in) the animal residence. In particular, the rearing stage of the animal may determine which microbes may be particularly beneficial, as well as which microbes may be particularly detrimental for the animal. Hence, in embodiments, the control system 300 may be configured to control the light generating device 100 in dependence of information from an animal rearing management system. In practice, some animals, such as adult chicken and chicks, may be commonly kept together despite being in different rearing stages. Hence, in embodiments, the control system may be configured to control the light generating device 100 in dependence of the (different) rearing stages of a plurality of animals.

In the depicted embodiment, the system 1000 further comprises a microbe dispenser device 400 configured to provide an emission 407 of the first microbes 7, especially to (an indoor space 3 of) the animal residence 200. In particular, the system 1000, especially the control system 300, may have a microbic emission mode, and the microbe dispenser device 400 may especially be configured to provide the emission 407 of the first microbes 7 in the microbic emission mode.

In embodiments, the control system 300 may be configured to control the microbic emission mode, especially the microbe dispenser device, in dependence of the microbiome influencing parameter. For instance, the control system 300 may be configured to introduce or increase the presence of first microbes in the animal residence based on (a value of) the microbiome influencing parameter.

In further embodiments, especially in the microbic application mode, the microbe dispenser device 400 may (be configured to) provide a (microbe) emission 407 of first microbes 7, wherein the microbe dispenser device 400 has a microbe emission region (or range) 415, i.e., the microbe dispenser device 400 may be configured to provide an emission 407 of first microbes 7 to the microbe emission region 415.

In the microbic lighting mode, the light generating device 100 may (be configured to) provide a first beam 115 of first device radiation 111, wherein the microbe emission region 415 and the first beam 115 may at least partly spatially overlap, such as for at least 30% of the microbe emission region 415, especially for at least 50%.

In the depicted embodiment, (in the microbic application mode) the microbe dispenser device 400 may (be configured to) provide a spray 410 of the first microbes 7.

The microbe dispenser device 400 may provide the first microbes 7 in an average direction E₁. The average direction may be a direction obtained by determining the direction wherein on average the most first microbes propagate away from the microbe dispenser device 400. For instance, the spray direction may be the average direction in which the first microbes propagate away from a spray microbe dispenser device.

Hence, in embodiments, the light generating device 100 may be configured to provide in a standard lighting mode white first device radiation 111, wherein the light generating device 100 is configured to provide in the microbic lighting mode white first device radiation 111; wherein a relative spectral power distribution of a first wavelength range relative to the spectral power distribution in the wavelength range of 200-780 nm, is higher during at least part of the microbic lighting mode than during at least part of the standard lighting mode. In further embodiments, the first wavelength range comprises the range of 405 nm+/−5 nm. In further embodiments, the first wavelength range comprises the range of 460 nm+/−5 nm.

In embodiments, the microbic lighting mode may temporally overlap with the microbic emission mode or may be temporally subsequent to the microbic emission mode.

The microbic application mode, the microbic lighting mode, and the standard lighting mode may (partially) overlap (in time).

In embodiments, the control system may be configured to select (and execute) one or more of the microbic application mode, the microbic lighting mode, and the standard lighting mode in dependence on the related input signal.

FIG. 1B schematically depicts an embodiment of a temporal arrangement of the microbic application mode, the microbic lighting mode, and the standard lighting mode over time T, wherein M1 refers to the microbic lighting mode, M2 refers to the microbic application mode, and M3 refers to the standard lighting mode. In a first phase I, the system may be in the standard lighting mode. Subsequently, in a second phase, the system may simultaneously be in the microbic application mode and the microbic lighting mode, i.e., the microbe dispenser device 400 may provide an emission 407 of first microbes 7, and the light generating device 100 may provide a first beam 115 of first radiation 111 to promote the persistence of the first microbes 7, especially relative to second microbes. At the end of the second phase II, the microbic application mode may cease until about ⅕^{th} of the way through a third phase III, during which the microbic lighting mode is continuously active, and during which the microbic application mode is partially active. At the end of the third phase III, the microbic lighting mode may switch off, and the standard lighting mode may be started for the fourth phase IV.

For instance, phases I may correspond to the end of a (working) day, during which standard lighting is used. At the end of the (working) day, such as after people leave the indoor space 3, the second phase II may begin and the system 1000 may initiate the microbic application mode and the microbic lighting mode. During phases II and III the system 1000 may prepare the indoor space 3 for the next (working) day. As indicated in the embodiment, the microbe emission 407 of first microbes 7 may be provided multiple times. For example, the system 1000 may during phase II providing a first set of first microbes 7 and may during phase III provide a second set of first microbes 7, such as a second set of first microbes belonging to different genera than the first microbes 7 of the first set. This could, for example, be relevant if the different first microbes 7 may be beneficially cultivated with first radiation 111 having different first spectral distributions. Or, for example, if the first microbes 7 of the second set depend on the first microbes 7 of the first set, which may, in such embodiments, first be allowed to settle in the indoor space 3. As the next (working) day starts, in phase IV, the system 1000 may again switch to standard lighting.

In further embodiments, the system may also execute the microbic lighting mode while the indoor space 3 is in use (by a human user).

In the depicted embodiment, the microbic lighting mode overlaps in time with the microbic application mode. In further embodiments, the microbic lighting mode may be arranged subsequent in time to the microbic application mode.

FIG. 1C schematically depicts a spectral power distribution for the standard lighting mode M3 and for the microbic lighting mode M1 in intensity I vs. wavelength λ. In particular, in the depicted embodiment, a relative spectral power distribution of a first wavelength range relative to the spectral power distribution in a reference wavelength range, such as the wavelength range of 200-780 nm, is higher during at least part of the microbic lighting mode M1 than during at least part of the standard lighting mode M3. In particular, in the depicted embodiment, the microbic lighting mode M1 may have an additional peak in the spectral power distribution at the left side of the distribution.

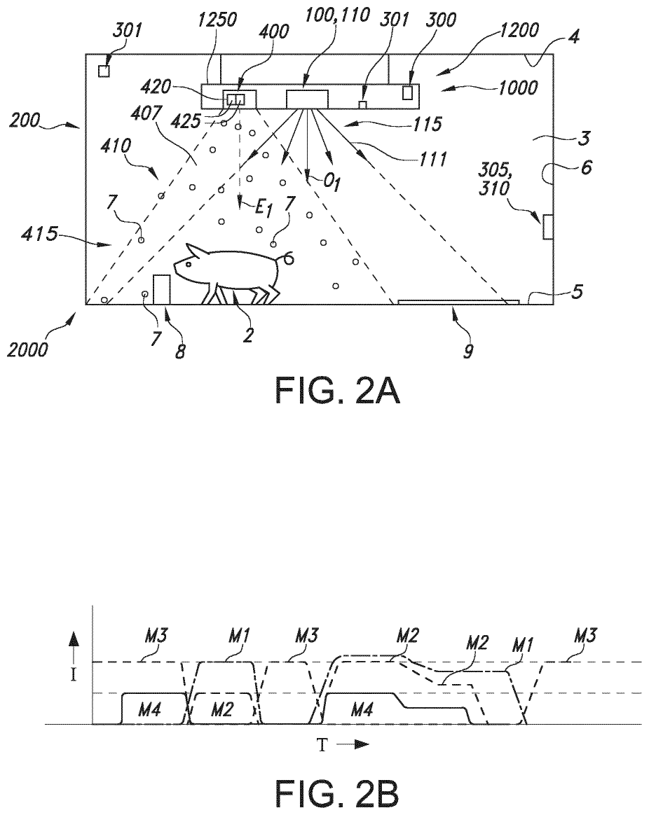

FIG. 2A schematically depicts a further embodiment of the system 1000. In the depicted embodiment, the light generating device 100 may in the disinfection mode (be configured to) provide disinfection radiation 121, especially wherein the disinfection radiation 121 comprises UV radiation. In further embodiments, the control system 300 may be configured to control the first device radiation 111, the disinfection radiation 121, and the emission 407 of the first microbes 7, especially in dependence of the related input signal, especially in dependence of the microbiome influencing parameter.

In embodiments, the input system 305 may comprise a sensor 310 selected from the group comprising a movement sensor, a presence sensor, an activity detection sensor, a people counting sensor, a distance sensor, an ion sensor, a gas sensor, a volatile organic compound sensor, a pathogen sensor, an airflow sensor, a sound sensor, a temperature sensor, and a humidity sensor, wherein the related input signal comprises a related sensor signal, i.e., wherein the input system 305 is configured to provide (a related input signal comprising) a related sensor signal to the control system 300.

In the depicted embodiment, the light generating device 100 may be configured to provide the first radiation 111 centered along a first optical axis O1, such as in a cone-shape centered along the first optical axis O₁, and the disinfection radiation 121 centered along a second optical axis O2. In particular, the light generating device 100 may be configured to provide the disinfection radiation centered along the second optical axis O2 in a first direction, indicated by O2′ towards an upper air space, especially towards the ceiling 4, or in a second direction, indicated by O2″, towards the floor 5. In further embodiments, the control system 300 may, for instance, control the disinfection radiation in the second direction in dependence of a signal from a sensor 301, such as of a presence sensor. Thereby, the system 1000, especially the control system 300, may avoid providing the disinfection radiation, especially comprising UV radiation, to an animal or a human in the (indoor space 3 of the) animal residence 200.

In the depicted embodiment, the light generating device 100 may be configured to provide the disinfection radiation towards an upper air space, which may be safely removed from potential people in the room. For instance, the upper air space may be the space in the (indoor space 3 of the) animal residence 200 above about 2.3 m.

Further, in the depicted embodiment, the light generating device 100 may be configured hanging from the ceiling 4. However, in further embodiments, the light generating device 100 may comprise a task light, such as a free floor standing luminaire or a light on a table.

In embodiments, the control system 300 may be configured to control a microbe emission rate of (the emission 407 of) the first microbes 7 in dependence of the disinfection radiation 121. Especially, one or more of the following may apply: (a) the microbic application mode and the disinfection mode may at least partly overlap in time, and relative to a baseline emission rate, the microbe emission rate during at least part of the disinfection mode is higher than the baseline emission rate; and/or (b) the microbic application mode and the disinfection mode may be temporally separated, and wherein relative to the baseline microbe emission rate, the microbe emission rate after the disinfection mode is higher than the baseline microbe emission rate. Hence, in embodiments, the microbe dispenser device 400 may be configured to increase a microbe emission rate during the disinfection mode in order to counteract negative effects of the disinfection radiation on the first microbes 7, or the microbe dispenser device 400 may be configured to increase a microbe emission rate after the disinfection mode in order to (re-)colonize the indoor space 3. In particular, in the latter embodiment, the microbe emission rate of the microbe dispenser device during the disinfection mode may be (essentially) 0, i.e., the microbe application mode may be temporally arranged (directly) after the disinfection mode.

In the depicted embodiments, the microbe dispenser device 400 may comprise a cartridge holder 420, especially wherein the cartridge holder 420 is configured to detachably host a plurality of cartridges 425. The plurality of cartridges may especially host (different) first microbes 7, such as to subsequently apply different first microbes as described above, and/or may comprise odors, especially odorous compounds. In further embodiments, two or more of the plurality of cartridges 425 may filled with material differing in one or more of types of first microbes 7 and types of odor, especially with two or more types of first microbes 7.

In further embodiments, the microbe dispenser device 400 may comprise a mist dispenser, especially wherein the mist dispenser is configured to dispense the first microbes 7.

In embodiments, the first beam 115 of first device radiation 111 may have a first direction V1 parallel to a first optical axis O1 of the first beam 115, and the second beam 125 of disinfection radiation 121 may have a second direction V2 parallel to a second optical axis O2 of the second beam 125, especially in a second direction indicated by O2″, wherein the first direction V1 and the second direction V2 have a mutual angle αM selected from the range of 90-180°. In the depicted embodiment αM may especially be about 180º.

In further embodiments, the lighting system 1000 may be configured to provide in a disinfection mode (of the lighting system 1000) one or more of disinfection radiation 121 and charged particles. In particular, in further embodiments, the disinfection radiation 121 may comprise one or more of (i) UV radiation having one or more wavelengths selected from the wavelength range of 100-380 nm, (ii) visible near UV radiation having one or more wavelengths selected from the wavelength range of 380-495 nm, and (iii) IR radiation having one or more wavelengths selected from the wavelength range of 750-950 nm.

In further embodiments, the system 1000 may comprise an ionizer device 130, wherein the ionizer device 130 is configured to provide the charged particles.

In embodiments, the control system 300 may have access to a predefined target microbiome composition, especially for (at least part of) the animal residence, wherein the control system 300 is configured to control the light generating device 100 in dependence of the microbiome influencing parameter and the target microbiome composition. In further embodiments, the control system 300 may be configured to control one or more of the microbic lighting mode, the microbic application mode, and the disinfection mode in dependence on the microbiome influencing parameter and the target microbiome composition. In particular, the control system 300 may be configured to steer a microbiome composition in (at least part of) the animal residence 200 towards the target microbiome composition.

FIG. 2A further schematically depicts an embodiment of a lighting device 1200 comprising the system 1000 In embodiments, the lighting device 1200 may further comprise a housing 1250, which may especially enclose at least part of the light generating device 100. In further embodiments, the housing may (also) enclose at least part of the microbe dispenser device 400.

In further embodiments, the lighting device may be selected from the group comprising a lamp, a luminaire, a projector device, a disinfection device, and an optical wireless communication device, especially a luminaire.

FIGS. 1A and 2A further schematically depict embodiments of the animal residence system 2000. In the depicted embodiment, the animal residence system comprises an animal residence 200 and the lighting system 1000. In particular, the lighting system 1000 may be configured to control the indoor microbiome in the animal residence 200.

In further embodiments, the animal residence 200 may comprise a rearing space 13, wherein the light generating device 100 of the lighting system 1000 is configured to provide the first device radiation 111 to the rearing space 13.

FIG. 2A further schematically depicts an embodiment of the method for indoor microbiome management in an animal residence. The method may comprise providing first device radiation 111 to (an indoor space 3 of) the animal residence 200, wherein a spectral power distribution of the first device radiation 111 is selected for promoting persistence, especially growth, of first microbes 7 relative to second microbes, other than the first microbes 7, wherein the method further comprises receiving and/or sensing, especially receiving, or especially sensing, a microbiome influencing parameter, and controlling (a spectral power distribution of) the first device radiation 111 in dependence of the microbiome influencing parameter.

In further embodiments, the method may comprise, in dependence of the microbiome influencing parameter, selecting the spectral power distribution for one or more of (i) promoting growth of the first microbes 7, (ii) deactivating the second microbes, other than the first microbes 7, and (iii) deactivating the second microbes more strongly than the first microbes 7.

In the depicted embodiment, the method may comprise providing in a microbic lighting mode a first beam 115 of first device radiation 111, and providing in a microbic application mode an emission 407, especially a spray 410, of the first microbes 7 in a microbe emission region 415. In particular, in the depicted embodiment, the first beam 115 and the microbe emission region 415 at least partly spatially overlap. In further embodiments, the microbic lighting mode may temporally overlaps with the microbic application mode, or may be subsequent in time to the microbic application mode.

In further embodiments, the method may comprise directing in a disinfection mode at least part of disinfection radiation 121 to a ceiling 4, especially wherein the disinfection radiation 121 comprises UV radiation.

In further embodiments, the method may comprise directing in the microbic lighting mode at least part of the first beam 115 of first device radiation 111 to a floor 5, and especially directing in the microbic application mode at least part of the emission 407 of the first microbes 7 to the floor 5.

FIG. 2B schematically depicts an embodiment of the method, and an embodiment of an operational mode of the system, wherein intensity I of radiation employed in different modes is very schematically depicted over time T. In particular, M1 may refer to the microbic lighting mode, M2 refers to the microbic application mode, M3 may refer to the standard lighting mode, and M4 may refer to the disinfection mode. By way of example, several situations are depicted over time, which do not necessarily occur one after the other, but are only depicted in a single drawing by way of comparison.

The drawing starts on the left with a standard lighting mode M3, e.g. white light, but also a disinfection mode M4. Due to the latter, microbes may be treated detrimentally. No microbes are applied yet.

Then, in a second stage, the standard lighting mode M3 changes into a microbic lighting mode M1, e.g. by adding intensity in the desired spectral range. If desired, the spectral power distribution may be changed a bit further, to obtain essentially the same color point and/or CRI even though the intensity in the desired spectral range is added. About at the same time, the microbic application mode M2 starts. Hence, now the room is substantially treated in the first state to reduce microbes (first and second microbes), now desired microbes are added, together with the beneficial light. Hence, the spectral power distribution of the first device radiation is selected for promoting persistence of the first microbes relative to second microbes.

When the desired situation is reached, the microbic application mode M2 may in the next stage be terminated, and the microbic lighting mode M1 may also be changed into the standard lighting mode M3.

In the fourth, longer stage it is suggested that at the same time the microbic application mode M2 and the disinfection mode M4 is applied. Hence, while undesired second microbes may de detrimentally treated, desired first microbes may be relatively promoted. By way of example the microbic lighting mode M1 is drawn at a higher intensity level than in the second stage, just to indicate by way of example that due to the disinfection mode M4, it may be desirable to stronger promote the first microbes over the second microbes by the microbic light in the microbic lighting mode M1. Further, by way of example in this first stage, some intensity levels are changed over time. Again, when the desired situation is reached, the microbic application mode M2 may be terminated, and the microbic lighting mode M1 may be changed into the standard lighting mode M3.

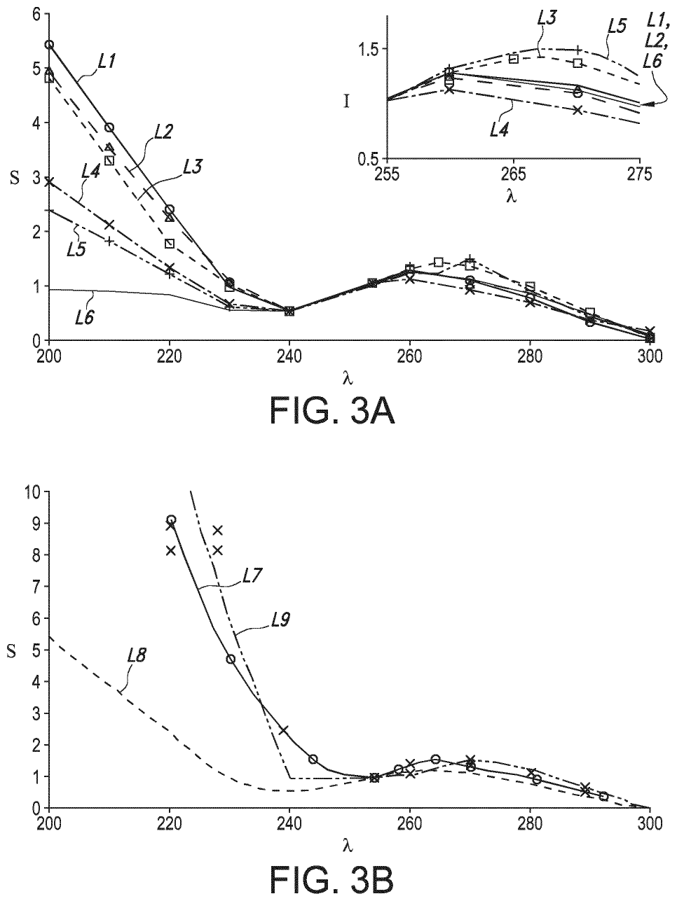

FIG. 3A-B schematically depict relative spectral sensitivities S versus wavelength λ (in nm). In particular, line L1 corresponds to MS2, line L2 corresponds to QB, line L3 corresponds to TIUV, line L4 corresponds to T7m, line L5 corresponds to T7 Coliphages, line L6 corresponds to C. parvum, line L7 and the crosses correspond to Bacillus pumilis, line L8 corresponds to MS2, and line L9 corresponds to Adenovirus. Data points at 200 and 300 nm are extrapolated.

The term “spectral sensitivity” may herein especially refer to the relative absorption of photons at the given wavelength, which photons may damage the microbes. As indicated above, microbes may further differ in their ability to repair UV-induced damaged, such as via DNA-repair mechanisms. Hence, in embodiments, the control system may select the spectral power distribution, and especially also the irradiance, of the first device radiation and/or of the disinfection radiation based on spectral sensitivities and repair mechanisms of the first microbes and the second microbes.

Hence, as illustrated in FIG. 3A-B, different microbes may have different spectral sensitivities for different wavelengths, which may facilitate providing first device radiation that provides first microbes a competitive advantage over second microbes, other than the first microbes. In particular, the differences in the relative spectral sensitivity between the different microbes may be most pronounced in the 260 nm-280 nm range (with most difference at 270 nm) and for UV wavelengths below 240 nm.

The term “plurality” refers to two or more. Furthermore, the terms “a plurality of” and “a number of” may be used interchangeably.

The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. Moreover, the terms “about” and “approximately” may also relate to 90% or higher, such as 95% or higher, especially 99% or higher, even more especially 99.5% or higher, including 100%. For numerical values it is to be understood that the terms “substantially”, “essentially”, “about”, and “approximately” may also relate to the range of 90%-110%, such as 95%-105%, especially 99%-101% of the values(s) it refers to.

The term “comprise” also includes embodiments wherein the term “comprises” means “consists of”.

The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

The term “further embodiment” and similar terms may refer to an embodiment comprising the features of the previously discussed embodiment, but may also refer to an alternative embodiment.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, “include”, “including”, “contain”, “containing” and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings. Moreover, if a method or an embodiment of the method is described being executed in a device, apparatus, or system, it will be understood that the device, apparatus, or system is suitable for or configured for (executing) the method or the embodiment of the method, respectively.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A lighting system for indoor microbiome management in an animal residence, wherein the lighting system comprises a light generating device, a control system, microbe dispenser device, and an input system, wherein:
the microbe dispenser device is configured (i) to provide in a microbic emission mode an emission of the first microbes;
the light generating device is configured to generate first device radiation, wherein a spectral power distribution of the first device radiation is selected for promoting persistence of first microbes relative to second microbes, other than the first microbes;
the input system is configured to receive and/or sense a microbiome influencing parameter and to provide a related input signal to the control system; and
the control system is configured to control the light generating device in dependence of the related input signal.

2. The lighting system according to claim 1, wherein the input system comprises a sensor, wherein the sensor is configured to sense the microbiome influencing parameter, and wherein the related input signal comprises a related sensor signal.

3. The lighting system according to claim 1, wherein the spectral power distribution of the first device radiation is selected for one or more of (i) promoting growth of the first microbes, (ii) deactivating second microbes, other than the first microbes, and (iii) deactivating second microbes more strongly than the first microbes.

4. The lighting system according to claim 1, wherein the control system is configured to control one or more of the spectral power distribution of the first device radiation, a duty cycle of the first device radiation, a dynamic lighting effect of the first device radiation, a spatial direction of the first device radiation, and the intensity of the first device radiation in dependence of the related input signal.

5. The lighting system according to claim 1, wherein the microbiome influencing parameter is selected from one or more of (a) a presence of first microbes and (b) a presence of second microbes.

6. The lighting system according to claim 1, wherein the microbiome influencing parameter is selected from the group of temperature, relative humidity, moisture level of a floor surface, a light from a source other than the light generating device, ventilation, and a ground cover material in the animal residence.

7. The lighting system according to claim 1, wherein the microbiome influencing parameter is selected from the group of type of animal, rearing stage of the animal, condition of the animal, feed for the animal, spatial density of the animal, activity of the animal, cleansing of the animal residence, treatment of the animal, addition/exchange of the animal, human activity in the animal residence, stress level of the animal, and fear behavior of the animal.

8. The lighting system according to claim 1, wherein the first microbes are selected from the group comprising Acinetobacter, Alcaligenes, Arthrobacter, Azospirillum, Azotobacter, Bacillus, Beijerinckia, Caldiarchaeum, Cenarchaeum, Deinococcus, Enterobacter, Erwinia, Flavobacterium, Lactobacillus, Nitrosoarchaeum, Nitrosocaldus, Nitrosomonas, Nitrosopumilus, Nitrosospira, Rhizobium and Serratia.

9. The lighting system according to claim 1, wherein the spectral power has an intensity at one or more wavelength selected from a first wavelength range of 405 nm+/−5 nm or from a second wavelength range of 460+/−5 nm.

10. The lighting system according to claim 1, wherein the microbic lighting mode is temporally subsequent to the microbic emission mode.

11. The lighting system according to claim 1, wherein the lighting system is configured to provide in a disinfection mode one or more of (a) disinfection radiation, wherein the disinfection radiation comprises one or more of (i) UV radiation having one or more wavelengths selected from the wavelength range of 100-380 nm, (ii) visible near UV radiation having one or more wavelengths selected from the wavelength range of 380-495 nm, and (iii) IR radiation having one or more wavelengths selected from the wavelength range of 750-950 nm, and (b) charged particles.

12. (canceled)

13. The lighting system according to claim 1, wherein the control system has access to a predefined target microbiome composition, and wherein the control system is configured to control the light generating device in dependence of the microbiome influencing parameter and the target microbiome composition.

14. A lighting device selected from the group of a lamp, a luminaire, a projector device, a disinfection device, and an optical wireless communication device, comprising the lighting system according to claim 1.

15. Animal residence system comprising (i) an animal residence and (ii) the lighting system according to claim 1, wherein the lighting system is configured to control the indoor microbiome in the animal residence.
